(19) United States
(12) Patent Application Publication
(10) Pub. No.: US 2025/0389699 A1
(43) Pub. Date: Dec. 25, 2025

(54) MULTI-STAGE FLUID CONDITIONING DEVICE AND METHOD

(71) Applicant: MAYEAUX HOLDING, LLC, Gonzales, LA (US)

(72) Inventor: VALMOND JOSEPH SAINT AMANT, III, ST AMANT, LA (US)

(21) Appl. No.: 19/309,266

(22) Filed: Aug. 25, 2025

Related U.S. Application Data
(63) Continuation-in-part of application No. 18/672,098, filed on May 23, 2024.

(51) Int. Cl. G01N 30/32; G01N 1/22; G01N 1/38

(52) U.S. Cl. CPC G01N 30/32; G01N 1/2247; G01N 1/38; G01N 2001/382

(57) ABSTRACT

A multi-stage fluid conditioning device formed to receive a series of drop-in modular conditioning components, the exemplary embodiment in the form of single-stage regulators, is provided for stepped pressure reduction in radial and stacked configurations, the system providing enhanced efficiencies including reduced footprint as well as easy access to individual stages for repair, maintenance or reconfiguration, even in the field, via the provision of an exterior access port for each conditioning component in the device in alternative configurations, the present device further providing enhanced energy efficiencies, decreased cost of implementation, and significantly reduced complexity, when compared to prior systems.

### DOMESTIC PRIORITY AS CLAIMED BY APPLICANT

The present application is a continuation-in-part of U.S. Utility patent application Ser. No. 18/672,098 filed May 23, 2024 entitled MULTI-STAGE REGULATOR FOR WET GAS SAMPLING AND METHOD, listing Valmond Joseph St Amant, III as inventor.

### FIELD OF THE INVENTION

The present invention relates to an improved system and method of sampling pressurized process fluids, and more particularly a system for on-line sampling of pressurized process gas having liquid entrained therein, otherwise known and referred to as multiphase or “wet”, including but not limited to Natural Gas or the like. The much-needed improvement of the present invention replaces the discrete vaporizer with a heated pressure regulator used therewith with a unique, multistage regulator with optional heater for providing an analytically-correct, vapor-only sample to an analyzer or the like utilizing a unique analytically-specific design.

The present invention is illustrated in a fluid conditioning device shown in the form of a multi-stage regulator comprising one or more conditioning components, which in the exemplary embodiments comprise drop-in pressure reducing components (also referenced for convenience as “pistons”) to provide staged pressure reduction, in a design optimized to be field serviceable. The present invention provides easily accessible, field-removable and replaceable modular conditioning components (in the exemplary embodiments shown as pressure reducing components) that are configured to be independently accessible for repair, or changed to provide different operating characteristics, without the necessity of disturbing the other components (or in this example, pressure reducing pistons). Further, the drop-in conditioning components (shown in the form of pressure reducing components) are designed for accessibility on-site, which can allow one to reconfigure, for example, transformation from a 4-stage regulator to a single-step regulator for single-staged pressure reduction or other configuration, with or without a heater option to limit Joule-Thomson effect cooling and associated condensation in wet gas or the like.

The exemplary embodiment of the present invention provides a multi-stage series of self-adjusting and self-controlling (that preferably do not require any preset calibration or spring/stem/seat adjustment . . . unlike and in contrast to U.S. Pat. No. 11,971,733) horizontal single-stage regulators to provide staged pressure reduction in a wet gas or like, each of the above providing enhanced efficiencies including reduced energy consumption, decreased cost of implementation or maintenance (the design providing more in-situ maintenance/servicing options), in a system having significantly enhanced thermal efficiencies coupled with significantly reduced complexity and size, when compared to prior systems. An optional self-limiting heater block heated option of an alternative embodiment of the present invention is designed specifically for analytical applications to vaporize a wet gas sample and provide up to 3 Liters/Minute to a gas analyzer (the maximum analytical flow rate required). Other devices can vaporize higher flow rates (up to 20 Liters/min) for non-analytical applications such as filling cylinders or powering gas fired heaters but necessarily have large internal volumes.

### BACKGROUND OF THE INVENTION

Natural Gas is comprised of a mixture of gases (See API 14.1 Section 6.3 and naturalgas.org). Natural Gas is bought and sold based on its heating value (BTU), which is derived from a compositional analysis of the Natural Gas. It is the BTU content that determines the monetary value of a given volume of Natural Gas.

To determine the total heat value of a given volume of gas, a sample of the gas is analyzed, and from the compositional data, its heat value per unit volume is calculated. This value is generally expressed in BTU/cu ft. The typical range of transmission quality gas ranges between 1000 and 1100 BTU/cu ft. Production gas, storage facility gas, NGL, and newfound Shale Gas can have much higher heating values up to or even exceeding 1500 BTU/cu ft.

There has been a long-standing controversy between gas producers and gas transporters regarding measurement of entrained liquid typically present in most high BTU/cu ft gas (rich or wet gas). The unique integral slice capillary probe used in the system of U.S. Pat. No. 10,690,570 allows the extraction of a gas sample containing entrained liquids for analysis of same.

Once extracted, to vaporize a sample comprising wet gas or gas containing entrained liquids, prior art systems (37 FIG. 9, 37′ FIG. 10) typically rely upon a vaporizer heated, monitored and controlled via a complex system comprising a Negative Temperature Coefficient (NTC) temperature sensor for feedback control, a cartridge heater or heater block, a temperature controller, and a thermal cut-off to prevent runaway temperatures should the controller or sensor fail. (see for example A+ Manufacturing, Inc January 2006 Genie Vaporizer Product Sheet). Following vaporization, the vaporized sample is sent to a heated regulator to reduce the sample pressure as required by the analyzer, which pressure reduction must be accomplished in a manner which prevents condensation occurring due to JT cooling.

An alternative to NTC heating blocks in prior art vaporizers also includes the use of Positive Temperature Coefficient (PTC) self-limiting BLOCK heaters, such as, for example, the INTERTEC SL BLOCKTHERM brand C24V Self-limiting Block Heater CSA 24, from Intertec Hess Gmbh, Neustadt/Donau, Barvaria/Germany.

Positive Temperature Coefficient (PTC) materials describes those that experience an increase in electrical resistance when their temperature is raised. Materials which have useful engineering applications usually show a relatively rapid increase with temperature, i.e. a higher coefficient. The higher the coefficient, the greater an increase in electrical resistance for a given temperature increase.

A PTC material can be designed to reach a maximum temperature for a given input voltage, since at some point any further increase in temperature would be met with greater electrical resistance. Unlike linear resistance heating or NTC materials, PTC materials are inherently self-limiting and do not require temperature sensors, controllers, or thermal cutoffs, so this material is useful for providing robust and uncomplicated heating elements or the like. PTC products typically may be used for 5-10 years before needing to be replaced.

There have been many applications of PTC heaters for diverse applications including EV car heaters, airplane floor panel heaters, water heater cores, even vaporizer heating elements for battery-powered vapes. A more relevant example of PTC material is the self-limiting heat trace cable such as Raychem (UK patent GB 2199451 A published 1988) as well as others. Non-PTC products like the standard electrical heater cartridges found in prior art heated regulators typically will only have a one-year warranty.

For example, the SL BLOCKTHERM brand C24v Self-Limiting Block Heater from Intertec of Neustadt/Donau, Germany uses PTC elements.

Once vaporized and pressure reduced via one or more pressure regulator stages, the sample has been typically transported to the analyzer by a tube bundle containing self-limiting heat trace cables and stainless-steel tubing.

Applicant's U.S. Pat. No. 10,126,214 teaches the use of Equations of State (EOS) phase diagrams (such as diagram D in FIG. 1) to help the user understand the limitations of prior art heated regulators (some even called vaporizing regulators, including the Applicant's prior art as well as third parties including for example, Tescom, GO, and Swagelok) when used with the Applicant's vaporizer. In some cases, the Applicant's prior art 4-stage heated regulator R′ as shown in FIG. 20 was utilized after the vaporizer to provide staged pressure reduction while avoiding excessive Joule-Thomson (JT) cooling and associated condensation risk, to maintain the vaporized sample in the gas phase region through the pressure reduction.

The 4-stage heated regulator R′ provided several unique approaches to solve the JT cooling problem by adding fixed pressure drops to the Applicant's prior art large capacity vaporizer and heated regulator in the form of staged consecutive, fixed area, piston components or the like which were not adjustable or designed to be field serviceable or adaptable, and was difficult to manufacture and assemble.

Applicant's U.S. Pat. No. 10,690,570 (the contents of which are incorporated herein by reference thereto) taught the use of pressure reducing components (also known as pressure cut) before the vaporization chamber. However, it has been found that in some cases, this technique can result in pre-vaporization flashing, disassociation or fractionation to certain multicomponent Natural Gas sample(s).

Others have incorporated metering valves and other restrictions before the vaporization chamber (See U.S. Pat. No. 11,525,761). While seemingly helping to control flow, it is believed that use of such valves or restrictions may result in pre-vaporization flashing of the sample across the valves, and fractionation of the sample before it enters the vaporization chamber of the device, which may result in inaccurate analysis of the BTU value of the sample. Even the addition of the small diameter loop passageway of incoming sample added to a vaporizer such as shown in U.S. Pat. No. 11,525,761 can be heated by the metal body of the device when it is placed inside a heated enclosure (See US 2023/0280246 A1 and US Patent RE47,478 E1).

Others have provided an alternative cold zone area thermal isolation gap of the vaporizer before the vaporization chamber (See U.S. Pat. No. 11,525,761), when compared to applicant's prior art vaporizer. The thermal isolation gap of the applicant's prior art vaporizer and that taught in the above '761 patent believed ineffective if the vaporizer is in a heated enclosure as, once the vaporizer is in a heated enclosure, the metal temperature becomes at least the enclosure temperature (possibly even the heater cartridge temperature) on both sides of the thermal isolation gap as well as the metal core through it.

The integral slice capillary probe (see for example Applicant's U.S. Pat. No. 10,690,570, there contents of which are incorporated herein by reference thereto) is an improvement to the system and method taught in Applicant's U.S. Pat. No. 10,126,214, for use when the two-phase pipeline fluid sample is not in the dense phase. The integral slice capillary probe can be used instead of bringing the fluid sample to the dense phase with pressure and/or temperature.

Applicant's U.S. Pat. No. 10,690,570 teaches the use of a self-limiting block heater instead of the conventional heater cartridge of the prior art vaporizer. However, the geometry of this solution is not believed conducive to replacing the position and location of the heater cartridge. To compensate for that problem, a brass rod or other thermally conductive rod was joined to the heater block, however, that solution did not include any regulation.

Lastly, U.S. Pat. Nos. 8,220,479, 8,616,228, and 9,588,024, the entire contents of which are incorporated herein via reference hereto, illustrate a conditioning assembly comprising a receiver formed to slidingly receive conditioning components which are stacked upon one another for serial flow therethrough (FIG. 20). Note the stacked configuration of piston component housings and associated pistons components, lacking exterior access for the individual stages, while designed for easy customization for use in diverse applications allowing the user to select the conditioning components from a collection of components offering different conditioning characteristics, including pressure regulation, to provide a customized fluid conditioning solution, can be challenging to maintain, service.

Particularly, the stacking arrangement of the conditioning components in the receiver as illustrated can make reconfiguring or repairing the various conditioning components stacked therein difficult, as it may require removal of the entire unit from the installation, and can required more or less complete disassembly of the device to access the one to several modular conditioning components (or even all components) to change, repair or remove even a single component (which can comprise, for example, a pressure regulation/reduction stage if the component comprises a regulator or pressure reducer). Accordingly, in-situ or field maintenance or reconfiguration of these devices may be considered a less-than-ideal task, due to the multiple parts and steps involved.

Accordingly, the prior art appears to fail to provide a custom configurable multi-stage conditioning device and method for wet gas or the like in a compact footprint, while also being configured to provide ready accessibility to the various stages so that the one can easily repair, service or reconfigure one or more stages in the field without complete disassembly or removal of the device, providing a more manageable and less time consuming field maintenance, repair, reconfiguration, or the like.

### GENERAL SUMMARY DISCUSSION OF THE INVENTION

The present invention provides needed improvement of the prior system, combining a adjustable pressure regulator in both single-stage and multi-stage drop-in modular conditioning capabilities, shown in the form of drop-in horizontal single-stage regulators (that preferably in the present embodiment do not require any preset calibration or spring/stem/seat adjustment) for pressure reduction, with simple and easy manufacturing and assembly and field customization and service, enhanced efficiency and smaller footprint than the prior art, reducing the space needed in the instrument enclosure, and thereby provide heretofore available space for additional equipment or upgrades as required. Analytical instrumentation space in the field is always a premium and many times is more valuable than money, which makes the compact footprint of the present system, when compared to prior art systems, that much more valuable.

Referring to FIGS. 14-16, in another exemplary embodiment of the invention, instead of metering valves or other restrictions before the regulator as shown in the general background discussion above, the present invention relies on a capillary flow path 60 of wet gas 63 from the sampling probe 61 to the regulator 62 to provide flow control without the need for any valves while eliminating the possibility of premature flashing and sample distortion, while inside the heated enclosure, greatly simplifying the cost for implementation and maintenance of the system.

The use of the integral slice probe with capillary tubing for sampling and providing flow to the regulator prevents disassociation and fractionation of the sample regardless of the enclosure temperature (hot or cold), delivering an analytically correct sample without the necessity of metering valves.

The Equations of State (EOS) phase diagram (such as diagram D in FIG. 1) discussed in the general background discussion of the present application can likewise be used with the present invention to determine when to use the more economical PTC self-limiting heater block and when the NTC cartridge heater, sensor, cut-off, and controller are needed.

The present invention is illustrated in the exemplary embodiments as a multi-stage regulator comprising one or more conditioning components, which in the preferred embodiment comprises “drop in” modular fluid conditioning (in this case, pressure reducing) components (referenced also as “pistons” for convenience) to provide staged pressure reduction, but in design optimized to be field serviceable, with each component being independently accessible by service personnel without the need to disturb the remaining stages.

A modular conditioning component—in the context of a piston-configured module for staged pressure conditioning—refers to a self-contained, interchangeable unit engineered to perform specific pressure adjustment or conditioning functions within a fluid regulator system. This component is designed in a piston configuration, meaning it uses a movable piston as the primary pressure control element.

For purposes of the present invention, “modular” is intended to indicate that the component is manufactured as an independent module that can be easily inserted (or “dropped in”) into a specifically designed, sealable receiver or cavity within the body of a regulator or other fluid conditioning apparatus. Such design enables quick replacements, maintenance, or upgrades without the need for major disassembly of the entire regulator. Accordingly, the modular conditioning component (also referenced as a “piston”) is optimized for easy replacement or system reconfiguration, supporting better serviceability than, for example, a stacked configuration or vertical, fully enclosed configuration, and precise pressure control in advanced regulator systems or the like.

When used for staged pressure conditioning, these unique modular conditioning components allow for pressure regulation to occur in steps (stages) as the fluid (gas or liquid) passes through multiple conditioning components within the regulator body. Each stage incrementally reduces or conditions the fluid pressure, often resulting in more stable or precise final delivery pressure—especially valuable in two-stage or multi-stage regulator designs where constant or controlled pressure is needed even as inlet pressure fluctuates.

The term “conditioning component” is not intended to be limiting as other components may likewise be used in the present invention, including sensors and monitoring components such as corrosion coupons, wireless monitoring devices such as temperature sensors (for example, thermistor sensors, thermometers, etc.) wireless monitoring devices, flow meters, pressure sensors, moisture sensors, gas sensors (e.g. H2S and others) liquid detectors, filters, etc. One functional commonality of the above components is that the fluid passing therethrough (which may comprise gas or a gas with liquid suspension or gas having entrained liquids (i.e., “wet gas”), or even liquid) interacts with each said component in some capacity, be it to, for example, condition (in the case of a phase separation membrane, pressure regulator, etc.) or provide data in a monitoring context (in the case of temperature sensors, flow meters, pressure sensors, etc.), so the term “interacted fluid” may be used to describe fluid which has passed through any of the above components.

The present invention would be particularly suitable for providing a series of pressure regulators in stepped reduction stages to limit or prevent JT cooling, as discussed herein. An example of the use of stepped pressure reduction using pressure regulators in series may be found in Mayeaux now U.S. Pat. No. 8,616,228, the content of which are incorporated herein by reference thereto.

It is stressed that the above component list is intended to be illustrative only, and not limiting, as there are many other conditioning/monitoring components which may likewise be used with the present system. The present invention provides a modularized system to readily assemble and easily customizable solution which is also easy to service, repair or reconfigure in the field, providing a solution to a long felt, but unresolved need in the industry for such a device.

The present invention thereby provides a less involved, more direct way to access individual stages as required, and in the present example, the pressure reducing pistons that can individually be selectively replaced for repair/maintenance/service etc., or changed to provide different a adjust or change the conditioning stages or other operating characteristics, without the necessity of disturbing the other components. Further, such a modular component can even be removed on-site and the compartment sealed, so as to field transform the device, for example, from a 4-stage regulator to a single-step regulator, to provide single-staged pressure reduction, or some other configuration.

With a heated option to limit Joule-Thomson effect condensation in wet gas or the like, the present invention provides a multi-stage series of regulators for staged pressure reduction, each of the above providing enhanced efficiencies including reduced energy consumption, decreased cost of implementation or maintenance (the design providing more in-situ maintenance/servicing options), in a system having significantly enhanced thermal efficiencies coupled with significantly reduced complexity and size, when compared to prior systems.

An optional self-limiting heater block heated option of an alternative embodiment of the present invention is designed specifically for analytical applications to vaporize a wet gas sample and provide up to 3 Liters/Minute to a gas analyzer (the maximum analytical flow rate required). Other devices can vaporize higher flow rates (up to 20 Liters/min) for non-analytical applications such as filling cylinders or powering gas fired heaters but necessarily have large internal volumes.

With its field configuration options and reduced manufacturing and assembly complexity, the present invention's embodiment is expected to provide the multi-stage regulator of the present invention an advantage over the aforementioned prior art systems.

The second embodiment heating means replaces the self-limiting heater block with a machined block containing an enhanced NTC heater receptacle with built-in controllers as shown below, if the EOS phase diagram shows the need for more precise and/or higher temperature control.

The aforementioned regulator can be mounted in a system which provides an analytically correct extraction and sample conditioning of the two-phase sample to be delivered to the analyzer.

While the exemplary embodiment of the present invention is presented in conjunction with the capillary probe described in U.S. Pat. No. 10,690,570, the exemplary usage is not intended to be limiting, as the invention as presented herein may be utilized with other sampling systems and methodologies.

In summary, with the integration of the regulator (including multi-stage regulator) into a single unit, as well as the utilization of a heated option (Heater Block or enhanced NTC unit), the present invention provides a heated regulator in a field-serviceable and much more compact footprint when compared to the prior art, resulting in the creation less required equipment and creation of valuable space 57 within the insulated closure 56 for other equipment or upgrades, as show in in FIGS. 11-13.

### BRIEF DESCRIPTION OF DRAWINGS

For a further understanding of the nature and objects of the present invention, reference should be had to the following detailed description, taken in conjunction with the accompanying drawings, in which like parts are given like reference numerals, and wherein:

FIG. 1 is a Soave-Redich-Kwong EOS chart, illustrating four phase regions of an exemplary hydrocarbon fluid stream sample composition, at varying pressure and temperature.

FIG. 2 is a frontal, side, cross-sectional, partially cutaway view of a first embodiment of the present invention, showing a single-stage vaporizer regulator with a PTC heater cartridge provided therein.

FIG. 3 is a side view of the invention of FIG. 1.

FIG. 4 is a side, partially-exploded view of the vaporizer of FIG. 2, showing the insertion of a heater cartridge into the vaporizer regulator body.

FIG. 5 is a bottom, partially cutaway view of an NTC heater cartridge which can be utilized with the system of FIG. 4

FIG. 6 is a bottom, partially cutaway view of a PTC heater cartridge which can be utilized with the system of FIG. 4.

FIG. 7A is a frontal, cross-sectional and partially cutaway, first side view of a multi-stage vaporizer regulator alternative embodiment of the invention of FIGS. 2-3, showing first, third and fourth regulator stages of the four total stages provided in the system, the present multi-stage regulator configured to provide stepped-pressure reductions so as to avoid or diminish Joule-Thomson effect condensation of a vaporized sample, the figure further illustrating the PTC heater cartridge of the present invention shown provided therein, as well as the passage from the vaporizer to the first regulator stage, and passage from the third regulator stage to the fourth regulator stage.

FIG. 7B is a frontal, cross-sectional and partially cutaway, second side view of the invention of FIG. 7A, illustrating the second and fourth regulator stages, the staged regulators configured to provide gradual, staged-pressure reductions so as to avoid or diminish Joule-Thomson effect condensation of a vaporized sample, the figure further illustrating the PTC heater cartridge of the present invention shown provided therein, and inlet and outlet passages.

FIG. 8 is a top, cross-sectional and partially cutaway view of the invention of FIG. 7, showing pressure reducers also referenced as modular regulator components 1-3 of the multi-pressure vaporizer regulator of FIGS. 7A-7B.

FIG. 8A is a top, exploded view of the invention of FIG. 7.

FIG. 8B is a side, cross-sectional view of a prior art modular regulator component.

FIG. 9 is a top, cutaway view of a prior art vaporizer system utilizing a separate heated regulator and required NTC controller.

FIG. 10 is a side cutaway view of the prior art system of FIG. 9.

FIG. 11 is a frontal, partial cut-away view illustrating an exemplary modular conditioning system in an insulated enclosure mounted to a pipeline including a capillary collection probe, the system utilizing a vaporizer regulator with PTC heater cartridge provided therein for heating, illustrating the reduced components required when compared to the prior art system illustrated in FIGS. 9 and 10.

FIG. 12 is a side, partially cut-away view of the invention of FIG. 11.

FIG. 13 is a top, close-up, partial view of the invention of FIG. 11, illustrating the vaporizer regulator component and free space available when compared to the prior art system illustrated in FIGS. 9 and 10.

FIG. 14 is a side view of the invention of FIG. 12 without the enclosure shown.

FIG. 15 is a perspective view of the invention of FIG. 14, without the enclosure shown.

FIG. 16 is a frontal view of the invention of FIG. 14, without the enclosure shown.

FIG. 17 is a frontal view showing the INTERTEC brand PTC option heater block for a prior art heated regulator.

FIG. 18 is a frontal view showing a prior art heated regulator with improved NTC heater block.

FIG. 19 is a cross-sectional view of the applicant assignee's prior art GENIE brand Heated Regulator (GHR) with pre and post heat exchange.

FIG. 20 is a side, partially cross-sectional view of the applicant assignee's prior art GENIE brand JTR multi-stage Heated Regulator, described in U.S. Pat. Nos. 8,220,479, 8,616,228, and 9,588,024, the contents of which are incorporated herein by reference thereto.

FIG. 21 is a side, partially cross-sectional view of applicant assignee's prior art vaporizer that utilizes a controller with built-in thermal cutoff.

FIG. 22 is a frontal, cross-sectional, first side view of a multi-stage regulator preferred embodiment of the present invention and alternative, third embodiment of the invention of FIGS. 2-8A, showing first, third and fourth regulator stages of the four total stages provided in the system, the present exemplary device in the form of a multi-stage regulator configured to provide stepped-pressure reductions so as to avoid or diminish Joule-Thomson effect condensation of a wet gas sample, including passage to the first regulator stage, and passage from the third regulator stage to the fourth regulator stage.

FIG. 23 is a frontal, cross-sectional, first side view of the invention of FIG. 22, illustrating the threaded covers removed from the opposing conditioning component or piston chamber openings for said first and third regulator stages.

FIG. 24 is a frontal, cross-sectional and partially cutaway, second side view of the invention of FIGS. 22-23, illustrating the second and fourth regulator stages, the staged regulators configured to provide gradual, staged-pressure reductions so as to avoid or diminish Joule-Thomson effect condensation of a vaporized sample, the figure further illustrating the inlet and outlet passages.

FIG. 25 is a frontal, cross-sectional, second side view of the invention of FIG. 24, illustrating the threaded cover and modular regulator component of the second stage removed from the piston chamber opening.

FIG. 26 is a frontal, cross-sectional, second side view of the invention of FIG. 25, illustrating the threaded cover of the second stage removed from the piston chamber opening, with the modular regulator component situated within said piston chamber.

FIG. 27 is a frontal, second side view of the invention of FIG. 25, illustrating the threaded cover and modular regulator component of the second stage removed from the piston chamber opening.

FIG. 28 is a frontal, cross-sectional, first side view of the invention of FIG. 22, illustrating the threaded covers and modular regulators removed from the opposing piston chamber openings for said first and third regulator stages, respectively.

FIG. 29 is a frontal, first side view of the invention of FIG. 28, illustrating the threaded covers and modular regulators removed from the opposing piston chamber openings for said first and third regulator stages, respectively.

FIG. 30 is a top, cross-sectional and partially cutaway view of the invention of FIGS. 22-29, showing pressure reducers also referenced as modular regulator components 1-3 of the multi-pressure vaporizer regulator.

FIG. 31 is an end view of the invention of FIGS. 22-30 with an exemplary self-regulating block heater mounted thereon with mounting bracket, illustrating a side view of the device of the present invention showing the second stage with cover thereupon and inlet opening situated below said second stage,

FIG. 32 is an upper, right-hand perspective view of the invention illustrated in FIG. 31.

FIG. 33 is a side, cross-sectional view of the invention of FIG. 31 showing exemplary heater block mounted to the device of the present invention.

FIG. 34 is a top view of an exemplary installation of the device of FIGS. 22-33 mounted for use with an NTC required controller in a sampling application.

FIG. 35 is a side view of the invention of FIG. 35.

FIG. 36 is a frontal, partial cut-away view illustrating invention of FIG. 35 situated in an insulated enclosure mounted to a pipeline including a capillary collection probe, the system utilizing a vaporizer regulator with PTC heater cartridge provided therein for heating, illustrating the reduced components required when compared to the prior art system illustrated in FIGS. 9 and 10.

FIG. 37 is a side, partially cut-away view of the invention of FIG. 36.

FIG. 38 is a frontal, cross-sectional, first side view of fourth alternative embodiment of the modular conditioning system of the present invention, illustrated in the form of a multi-stage regulator of the inventions of FIGS. 2-8A and FIGS. 22-30, the present figure showing first, second and third conditioning stages (illustrated in the form of regulator stages), each stage configured for independent accessibility of modular conditioning components situated therein via adjacent access openings, said serially-situated stages providing flow to a fourth adjustable regulator stage, so as to provide four stages, the present exemplary device in the form of a multi-stage regulator configured to provide stepped-pressure reductions so as to avoid or diminish Joule-Thomson effect condensation of a wet gas sample, including passage to the first regulator stage, and passage from the third regulator stage to the fourth regulator stage.

FIG. 39 is a frontal, cross-sectional, second side view of the multi-stage regulator of FIG. 38.

FIG. 40 is a cross-sectional, first side view of the invention of FIG. 38, illustrating the threaded covers and modular conditioning components (discussed in the form of regulators) removed from the stacked piston chamber openings for said first, second and third and third regulator stages, respectively.

FIG. 41 is an opposing, second side view of the multi-stage regulator of FIG. 40.

FIG. 42 is a cross-sectional, third side view of the multi-stage regulator of FIG. 39, with the modular conditioning components removed, illustrating stacked first, second and third modular component receivers formed to receive said first, second and third modular conditioning components (not shown).

FIG. 43 is an opposing, third fourth side vide of the multi-stage regulator of FIG. 42, illustrating the outer body and upper adjustable regulator (forming the fourth stage in the present embodiment), upper flow outlet port and lower flow inlet port.

FIG. 44 is a frontal, cross-sectional, first side view of an alternative embodiment of the modular conditioning system of FIGS. 38-43 showing stacked first, second and third conditioning stages (illustrated in the form of regulator stages), each stage configured for independent accessibility of modular conditioning components situated therein via adjacent access openings, said serially-situated stages providing flow to a fourth adjustable regulator stage, so as to provide four stages, the present exemplary device in the form of a multi-stage regulator configured to provide stepped-pressure reductions so as to avoid or diminish Joule-Thomson effect condensation of a wet gas sample, including passage to the first regulator stage, and passage from the third regulator stage to the fourth regulator stage, the present invention further including the use of alignment pins situated between the stacked components to ensure and maintain proper orientation of component receiver associated with each modular chamber component with respective access ports or openings.

FIG. 45 is a side view of a first modular chamber component of the invention of FIG. 44 with internal configuration shown in phantom, and further illustrating an alignment pin emanating from the top of said component, said pin formed to engage a pin socket formed in the bottom of the second modular chamber component to be stacked thereupon.

FIG. 46 is a side view of the spacer block component of the invention of FIG. 44 with internal configuration shown in phantom, and further illustrating an alignment pin emanating from the top of said component, said pin formed to engage a pin socket formed in the second end of the longitudinal receiver of said body to facilitate and maintain alignment of the stack of modular components as discussed herein.

FIG. 47 is a side view of the stack of the invention of FIG. 44 with internal configuration shown in phantom, and further illustrating use of the alignment pins between said stacked components to facilitate and maintain alignment of same.

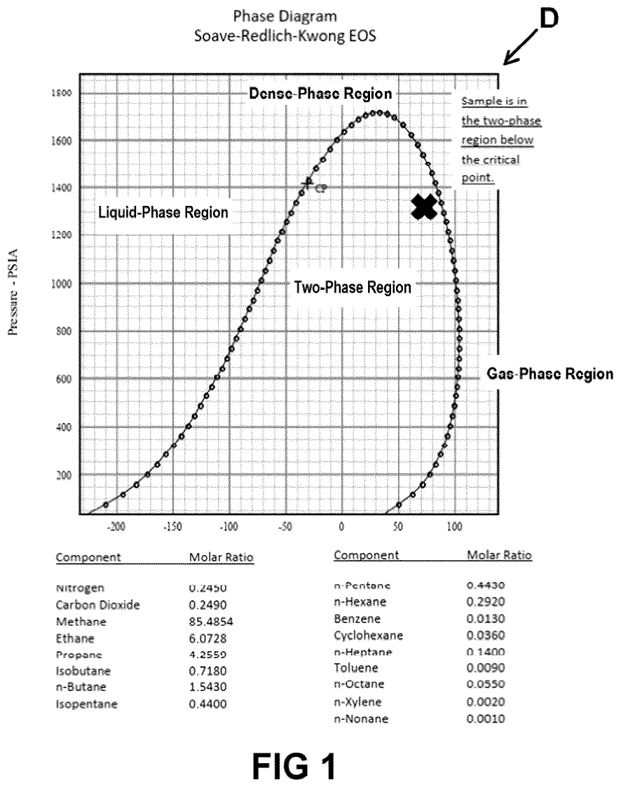

### DETAILED DISCUSSION OF THE INVENTION

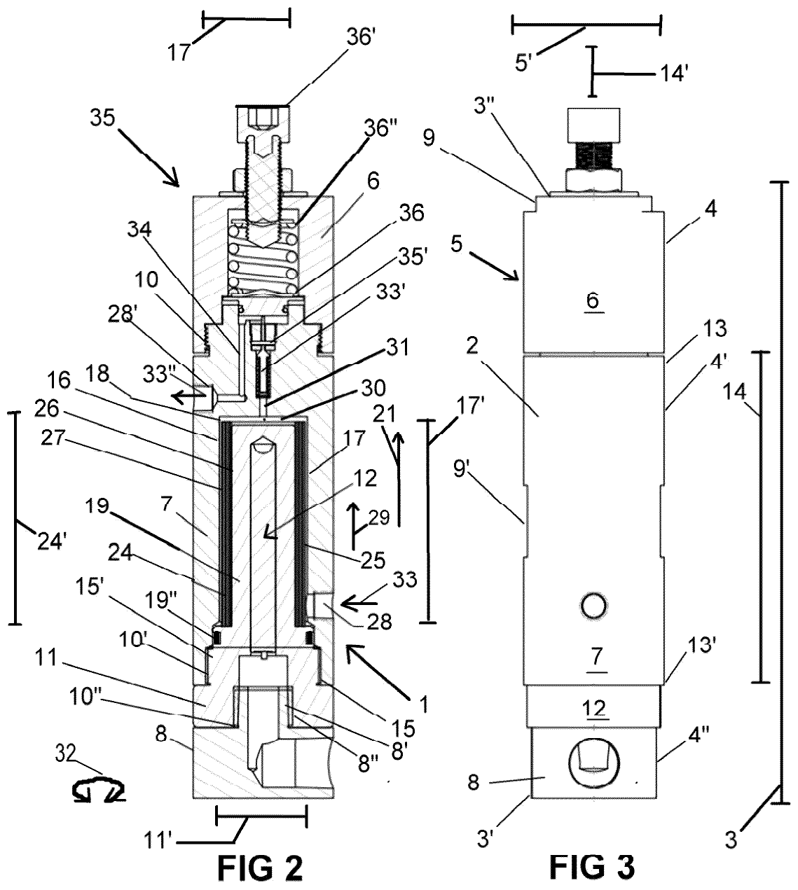

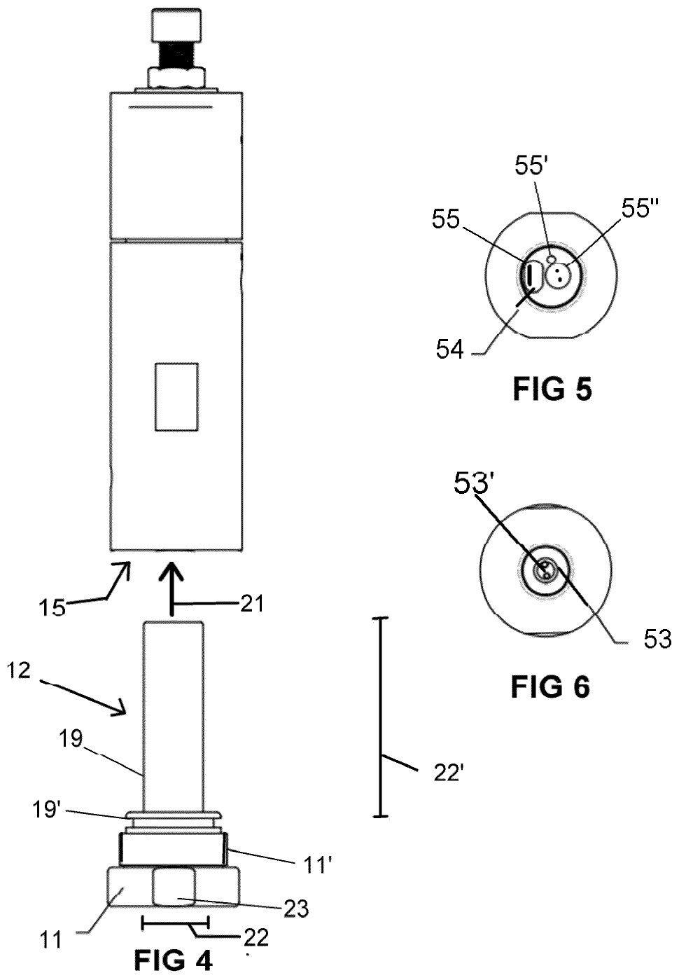

FIGS. 2-6 illustrate an exemplary embodiment of an adjustable single-stage vaporizing regulator 1, which has a body 2 having an overall length 3 defining first 3′ and second 3″ends, comprising threadingly 10, 10′, 10″ connected, first or upper 4 and second or lower 4′ sections containing an adjustable regulator 6 and vaporizer 7, the overall (assembled) device shown as having a cylindrical outer surface 5 defining an outer diameter 5′ with wrench flats 9, 9′ formed in the upper 4 and lower 4′ sections to facilitate engagement (and tightening or loosening) via an open-ended wrench or the like.

The lower section 4′ of the body 2 of the present invention has first 13 and second 13′ ends defining a length 14, the first 13 end having a receiver 16 formed therein along its length, said receiver 16 having an inner wall forming an inner diameter 17 and depth 17′ formed to receive 21, via threaded 15′ opening 15 at said second 13′ end of the vaporizer section 7, a heater cartridge 12.

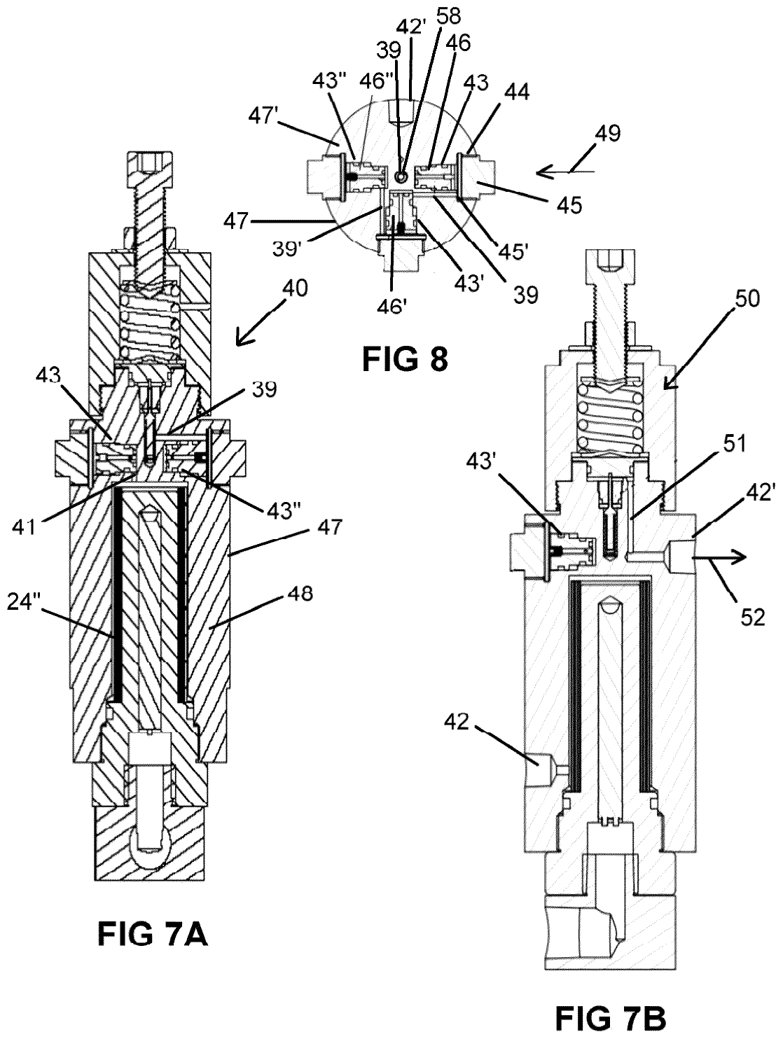

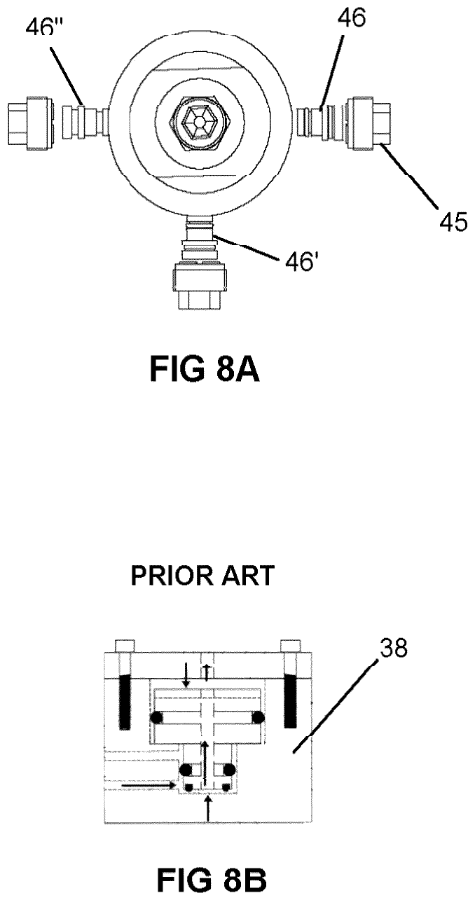

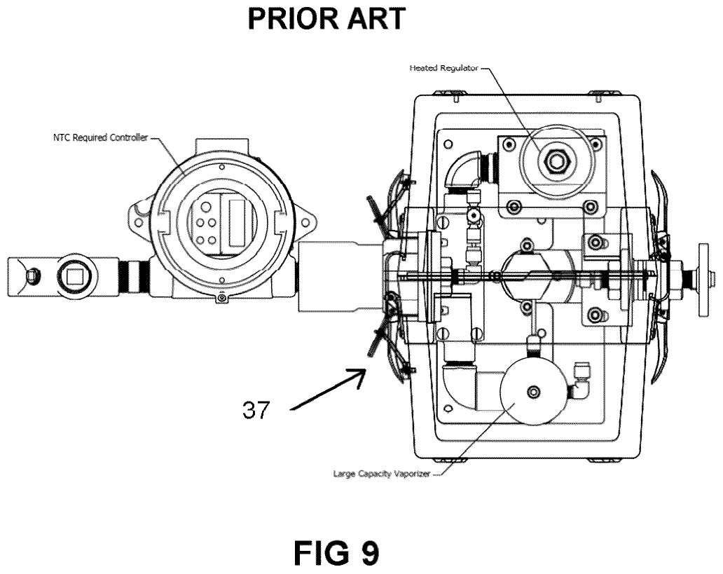

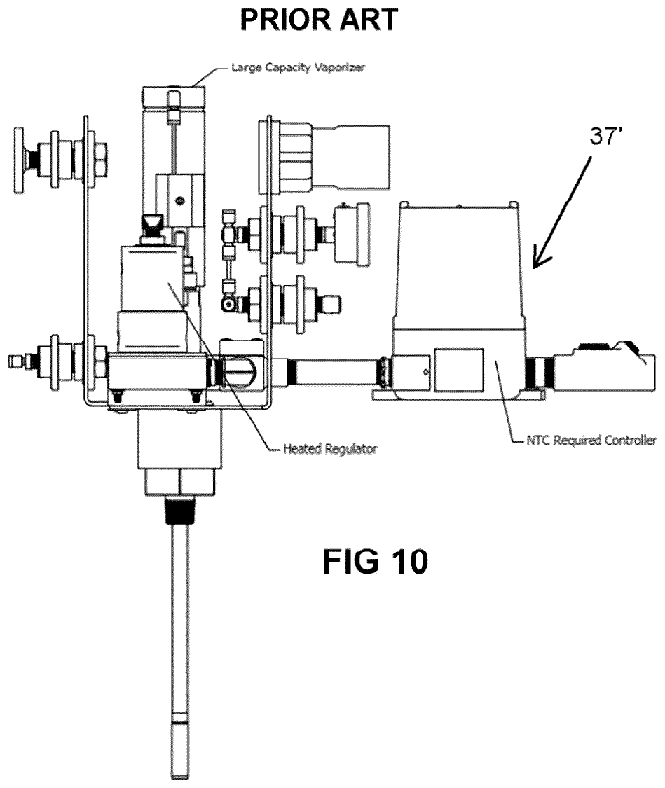

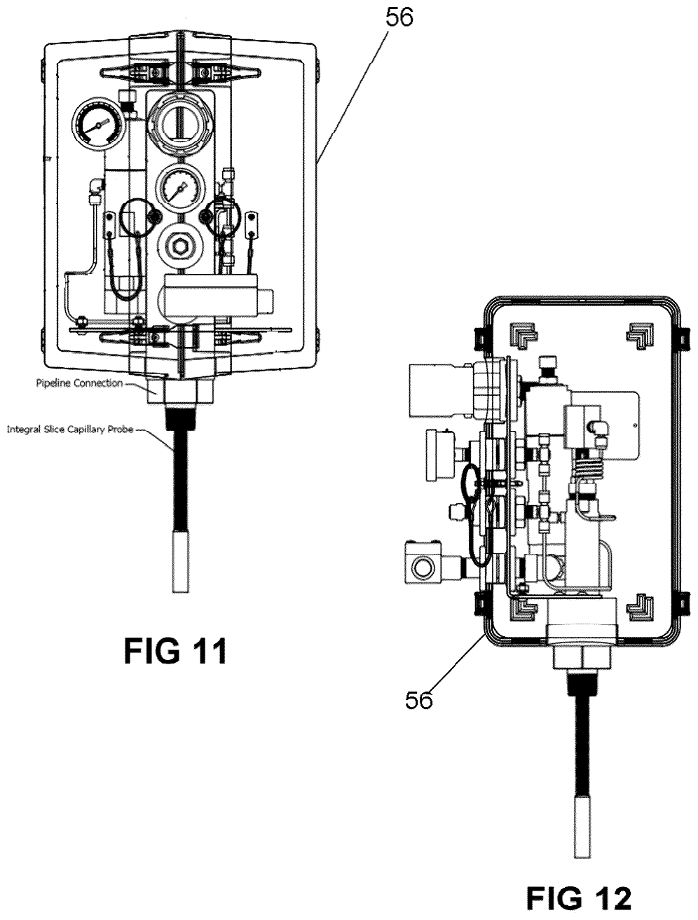

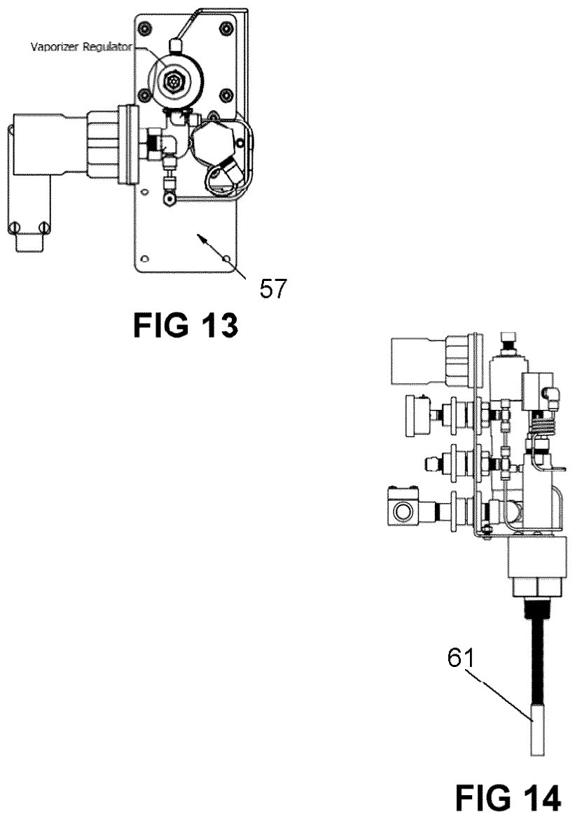

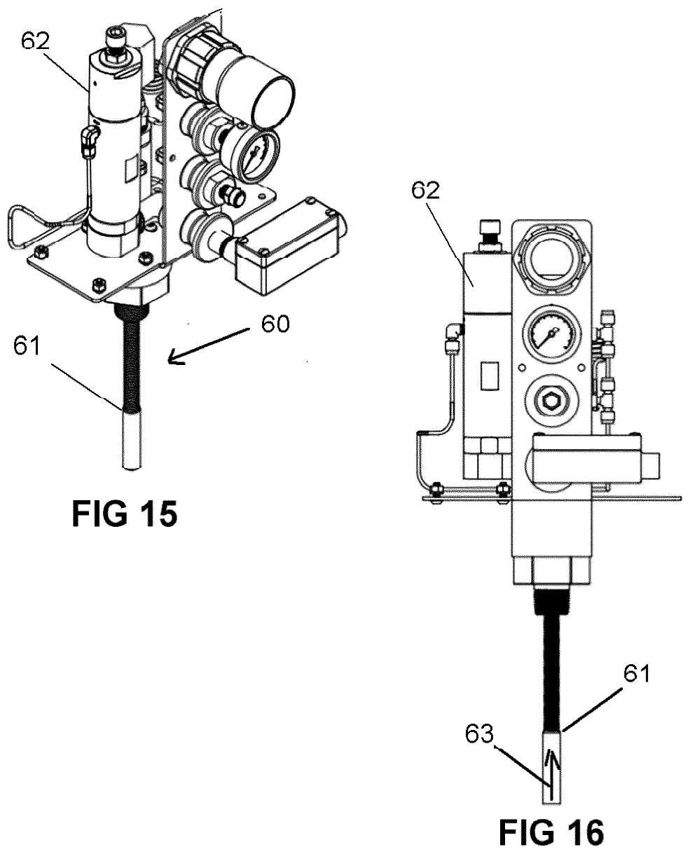

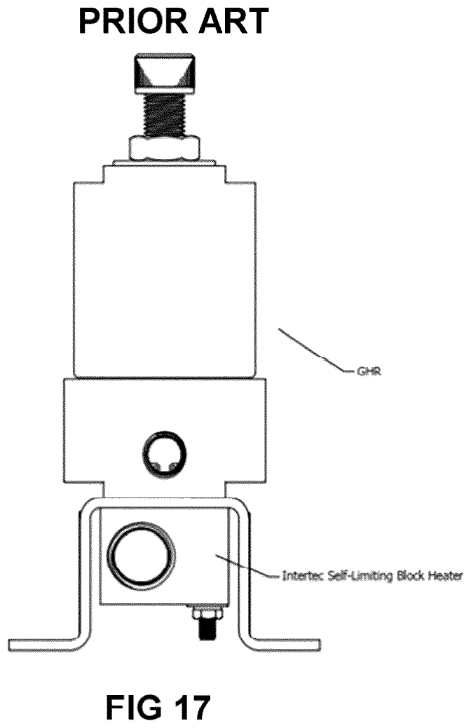

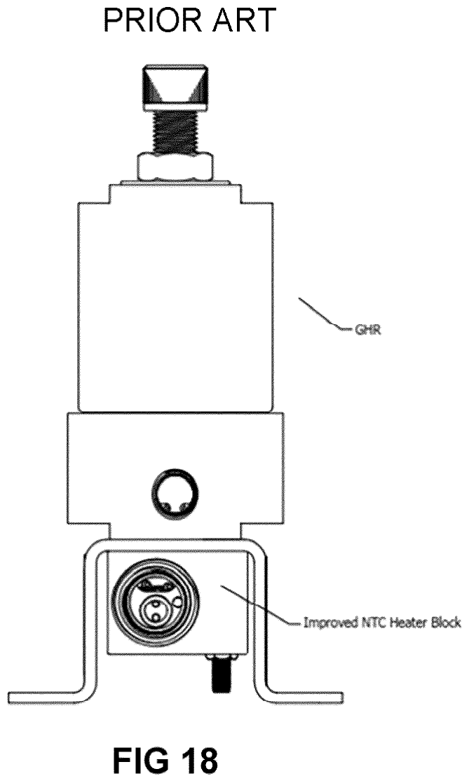

The heater cartridge 12 comprises a threaded 11′ base 11 having an outer wall forming an outer diameter 11′ with a groove 19′ for an O-ring 19″ for a sealed threaded connection with the vaporizer section, the base further having formed thereon and wrench flat 23. The heater cartridge 12 further comprises a thermal conductor sleeve 19 (an aluminum or stainless steel sleeve is used in the present example depending on the type of heater used, although other thermally conductive material could be used depending on the application and circumstances of use), having an outer diameter 22 and length 22′, the thermal conductor sleeve formed to contain therein a heater core, for example, a PTC heating element (FIG. 6) or enhanced NTC heating element (FIG. 5) as will be discussed herein, the depth 17′ of the receiver being sufficient to receive 21 the length 22′ of the thermal conductor sleeve 19 with heater core, so that the space 25 between the outer surface 26 forming the outer diameter 22 of the thermal conductor sleeve and the inner diameter 17 formed by the receiver 16 sidewall forms a vaporization chamber 24 having a length 24′ commensurate with the length 22′ of thermal conductor sleeve 19.

The heater core receives power via the conduit adapter, which engages a cylindrical socket 8″ formed in the heater core base 11 via cylindrical plug 8′ emanating from conduit adapter 8, to facilitate pivotal adjustment 32 as required for the device to have conduit approach from any direction as the adapter can be rotated 360 degrees. As earlier discussed, the PTC heater cartridge 53 is self-regulating so does not require a temperature sensor or cutoff, and includes a built-in power connector 53′ in its base, while the enhanced NTC heater cartridge 54 incorporates a built-in thermal cut-off 55 and temperature sensor 55′ in addition to the power connector 55″.

A screen or mesh 27 formed of thermal conducting material (for example, a Stainless Steel, 60 Mesh Screen roll formed to be situated in the space 25 or gap between thermal conductor sleeve and the receiver sidewall) is thereby provided in the space 25 between the outer diameter 22 of the thermal conductor sleeve enveloping the heater core, and the sidewall forming the inner diameter 17 of the receiver 16, the mesh 27 provided to facilitate enhanced heat transfer via the heater core and thermal conductor sleeve 22 outer surface, to fluid flowing 29 through the vaporization chamber 24.

A fluid inlet 28 is provided at about the second end 13′ of the body 2 of the vaporizer 7 section to receive a flow of fluid 33 (wet gas or the like) and direct same to the vaporization chamber 24 to vaporize entrained liquids or the like and facilitate fluid flow 29 therethrough (contacting thermal conducting mesh 27) utilizing heat emanating from the conductor sleeve 19 via the heater core to facilitate heat transfer to the fluid flowing through the passage forming the vaporization chamber 24, such that fluid flowing therethrough is heated as it traverses the length of the vaporization chamber 24, so as to facilitate the vaporization of any liquids entrained therein to gas, which gas flows out of the vaporization chamber via outflow passage 31 in the vicinity of the first end 13 of vaporizer body 2, via clearance 30 between the distal tip of the conductor sleeve, and distal end 18 of receiver 16 from opening 14. As shown, outflow passage 31 is situated along the longitudinal axis 13′ of vaporizer body 2, and situated between regulator section 6 and vaporization chamber 24.

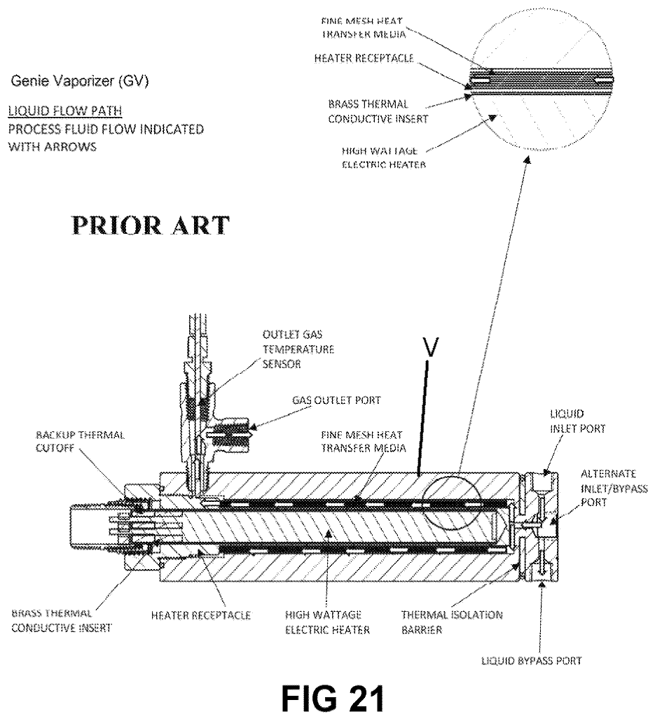

When compared to the applicant's prior art, GENIE brand vaporizer product (distributed by A+ Manufacturing Inc and shown in FIG. 21), the vaporizing area for the present device has a comparatively shortened length and does not utilize or require the thermal isolation section shown in the prior art vaporizer device V of FIG. 21

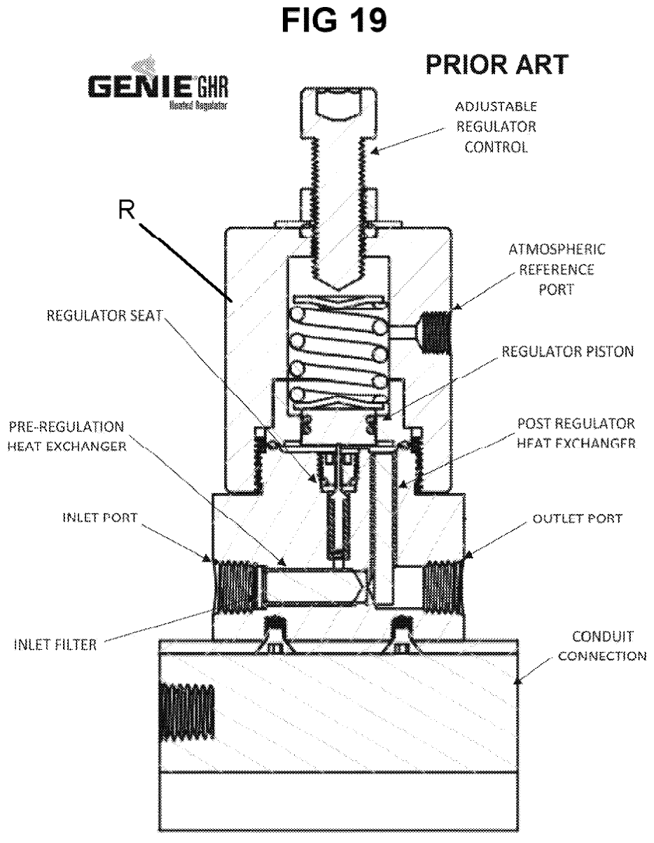

The vaporized sample 33′ leaves the vaporization chamber and flows via to the adjustable regulator section 6 where pressure reduction occurs, so as to provide a regulated, lower pressure sample. The adjustable regulator 35 shown in the present exemplary embodiment comprises a spring biased regulator seat 35′, piston 36 and adjustable mechanism 36′ in the form of a threaded bolt or the like to adjust spring bias of a helical compression spring 36″ applying bias on the piston 36, similar in operation of the prior art GENIE GHR brand heated regulator top regulator R section shown in FIG. 19.

The lower pressure sample then travels back down into the heated vaporizer portion of the body, for post-heat exchange passage 34 after regulation before flowing from the device via outlet 28′, which is situated between regulator section 6 and vaporization chamber 24 so as to receive residual heat from said vaporization chamber for post-heat exchange, and in parallel alignment with post heat passage 31.

Continuing with the Figures, an alternative embodiment of the single-stage adjustable regulator of FIGS. 2-3 is shown in FIGS. 7A-8A, which illustrate a novel and highly efficient, radially-configured multi-stage adjustable vaporizing regulator 40 having comprising a vaporizer 24 having the same elements and operating characteristics of the vaporizer of the embodiment of the single stage adjustable regulator embodiment of FIGS. 2-3, with the description of same incorporated herein via reference thereto. Of course, it is noted that the dimensions and other specifications regarding the vaporizer can vary depending on the operating characteristics and application.

Continuing with FIGS. 7A-8A, a post vaporizer passage 41 emanating from the vaporizer chamber 24″ opposite the inlet 42 provides a short passage of vaporized fluid to the first (stage 1) of four regulator stages, stages 1-3 in a radial positioning relative one another relative to a common center point 58, each stage each comprising piston chambers 43, 43′, 43″ formed to receive modular regulator components 46, 46′, 46″, respectively, the piston chambers 43, 43′, 43″ forming openings along the outer radial surface 47 of the cylindrical body 48 of the device, piston chambers 43, 43′, 43″ situated in serial, spaced relationship relative to one another along common radial plane 47′ and linked via a single passage 39, 39″ from one to the other in series, respectively.

Each piston chamber 43, 43′, 43″ has its own access port comprising a threaded opening 44, for receiving a threaded cover 45 with O-Ring 45′ or other seal to provide to a sealed chamber, each chamber sized to receive a modular conditioning component, shown in the present exemplary embodiment as modular regulator components 46, 46′, 46″ therein respectively, with each said each modular regulator component 46, 46′, 46″ inserted 49 or otherwise placed in to their respective piston chamber 43, 43′, 43″, and sealed therein via threadingly engaging a respective cover 45 with O-ring 45′ to the threaded opening 44 of each piston chamber.

Applicant assignee's patents U.S. Pat. No. 10,690,570 or U.S. Pat. No. 8,220,479B1, the contents of which are incorporated herein via reference thereto, teaches examples of modular single stage pressure ratio regulator (see for example 38 in FIG. 8b, which is from applicant's U.S. Pat. No. 10,690,570) illustrating the various components and operation which could be utilized in modular regulator component for the present application, although it is emphasized the item illustrated in FIG. 8 is provided to illustrate the general operational characteristics and elements, although the device would have to be configured and sized to fit the present application.

Each pressure reducing component in the form of a modular regulator in the present exemplary embodiment of the invention of FIGS. 7-8A would utilize a reduction piston or the like configured to provide a fixed pressure reduction, selected from a collection similar sized of modular regulators configured to provide various pressure cuts which could be inserted into the piston chamber(s) of the multi-stage regulator. Each modular regulator component of the present invention is selected to provide the desired pressure cut ratio for each stage. The configuration of the piston housing of a particular component may be customized to accommodate both the inside dimensions required to contain the required piston within the piston housing, as well as the outer dimensions required to fit within the chamber to which it to be inserted.

Each modular regulator component 46, 46′, 46″ comprises a piston housing which is formed to receive pressure reducing piston provided to reduce the pressure of wet gas flowing therethrough, providing a pressure cut of the fluid flowing into the vaporizer to facilitate the flash vaporization thereof, providing vaporized gas, which can then be further conditioned downstream, i.e., flowing to pressure regulator (such as an adjustable regulator) as further discussed herein.

Each modular regulator component 46, 46′, 46″ will provide a staged pressure drop or pressure cut of the fluid flowing therethrough, with multiple staged components in series, such as the present invention, to provide a more gradual pressure reduction, to diminish the excessive amount of cooling from taking place in single stage systems (many times the Joule-Thomson (JT) cooling that takes place when a gas is reduced in pressure).

A further advantage of the present “drop-in” modular component feature is that it allows ready customization of the desired pressure drop to facilitate the desired vaporization or other conditioning required, by choosing the appropriate specification pressure reducer component apparatus, which can offer different pressure reduction characteristics while maintaining similar exterior dimensions/configuration, but with different pressure reduction characteristics, allowing the user to choose the desired pressure reduction characteristic for the particular installation, and insert or “drop in” the appropriate pressure reducer component into the pressure reduction component receiver of the unit.

The first, second, third and fourth regulator stages, stages 1-3, being “drop in” modular regulator components and the fourth being manually adjustable thereby provide customizable, stepped-pressure reductions so as to avoid or diminish Joule-Thomson effect cooling and associated condensation of the vaporized sample flowing therethrough, the figure further illustrating utilization of the PTC heater cartridge of the present invention shown provided therein, as well as the passage from the vaporizer to the first regulator stage, and passage from the third regulator stage to the fourth regulator stage.

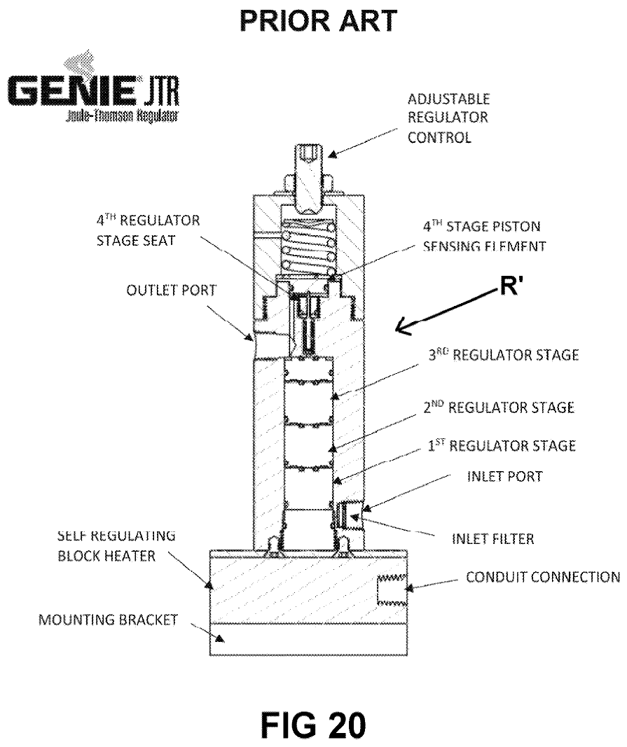

The radial design configuration of stage 1-3 modular regulator components 46, 46, 46″, respectively, of the present invention, are provided in a unique radial configuration situated along a radial plane 47′ just upstream the vaporizer, and below the upper 4 vaporizer body 2 (the body constructed of thermally conductive material such as stainless steel), and utilizes this position intermediate the vaporizer and fourth stage, to exploit the pre-heat characteristics of the treated fluid having just been heated via heater cartridge 12 as well as their physical proximity to same and associated heat zone (as well as exploiting the thermal conductivity of the body 2 to transfer heat from the vaporizer area to the regulator components) provides an enhanced thermal efficiency, as opposed to the prior art which relies upon heat from relatively distant heater block design such as shown in the prior art (for example, see FIG. 20).

Rather, the sample gas is exposed directly to the thermal conductive sleeve 19 or jacket heated by the heater cartridge 12 before reaching the pressure reduction stages, with the distance from the three staged pressure reduction stages and the heater cartridge 12 being minimal AND uniform as to stages 1-3, as opposed to the greater distance of the regulator(s) as provided in the prior art as well as non-uniform heat transfer in prior art due to increasing distance for each stage (for example see FIG. 20 applicant prior art).

The radial design of the present embodiment of the invention of FIGS. 7A-7B and 8 is more efficient for heat transfer than the stacked design of prior art FIG. 20 since the radial design provides uniformity as well as proximity to the heat source. Further, the radial design of the present invention provides easy access of each piston to allow for individual changes and/or maintenance as required, as opposed to having to require disassembly of the entire unit to change or repair an individual regulator stage, or replace O-ring seals as required on an individual basis as in various prior art systems.

In addition, one can simply remove a regulator and enclose the chamber via the threaded cover 45 with seal where reconfiguration, maintenance, or even removal of a particular stage, when a reduction stage is not required. Another type of conditioning or sensing component may be put in its place.

Following passage through the third piston chamber 43 and associated regulator component 46″ (when used), the reduced fluid then flows via passage 39 to the final, adjustable regulator 50 for a final pressure reduction or regulation, said adjustable regulator 50 having the same components and operation as the adjustable regulator of the single-stage, first embodiment of the invention shown in FIGS. 2-3, above, with the description of same is incorporated herein via reference thereto.

After passage through the final stage (the adjustable regulator 50), the pressure reduced, vaporized fluid 52 flows through passage 51, which passage directs the fluid back toward the heat source, so as to provide post regulator heat exchange of the fluid before it flows out of the device via outlet 42′, which is situated along the same radial plane 47′ as the stage 1-3 regulator components 46, 46′, 46″, respectively.

### Regulator with Radially-Configured Multi-Stage Serial Flow

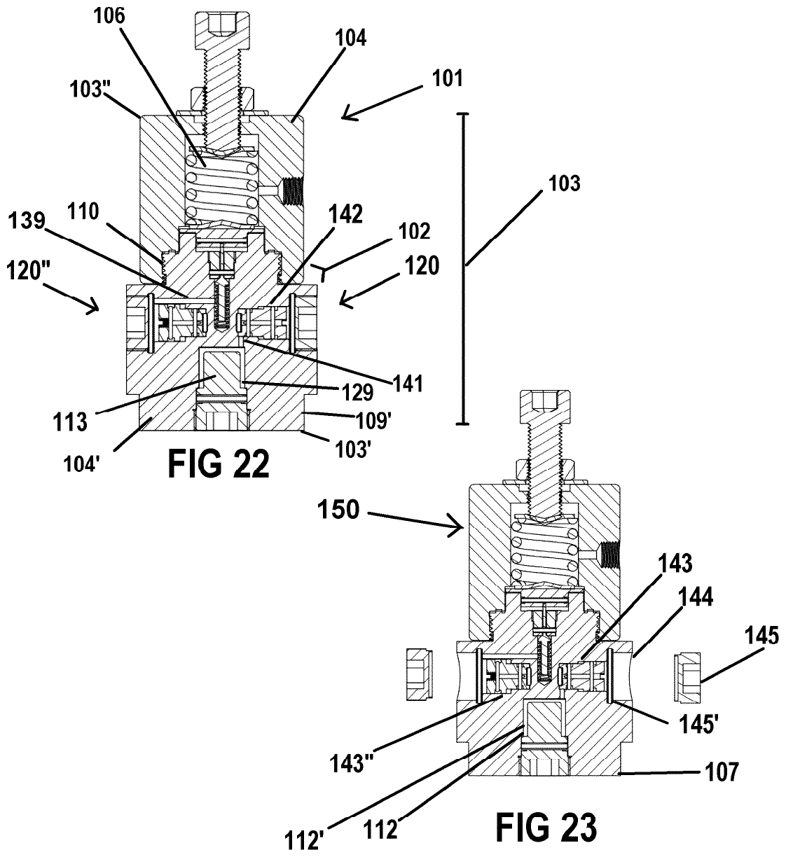

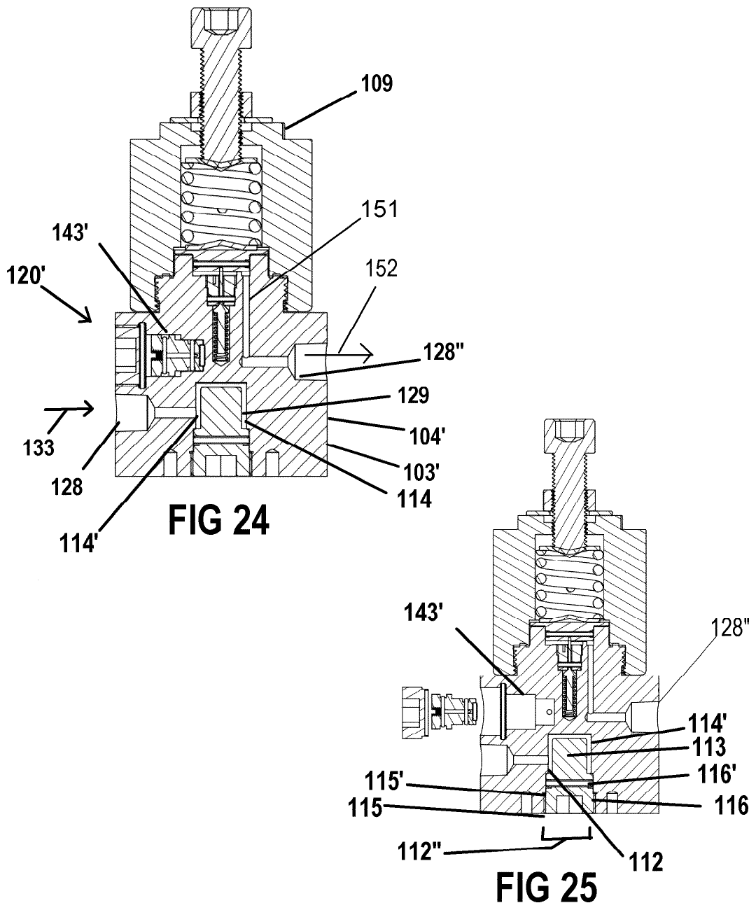

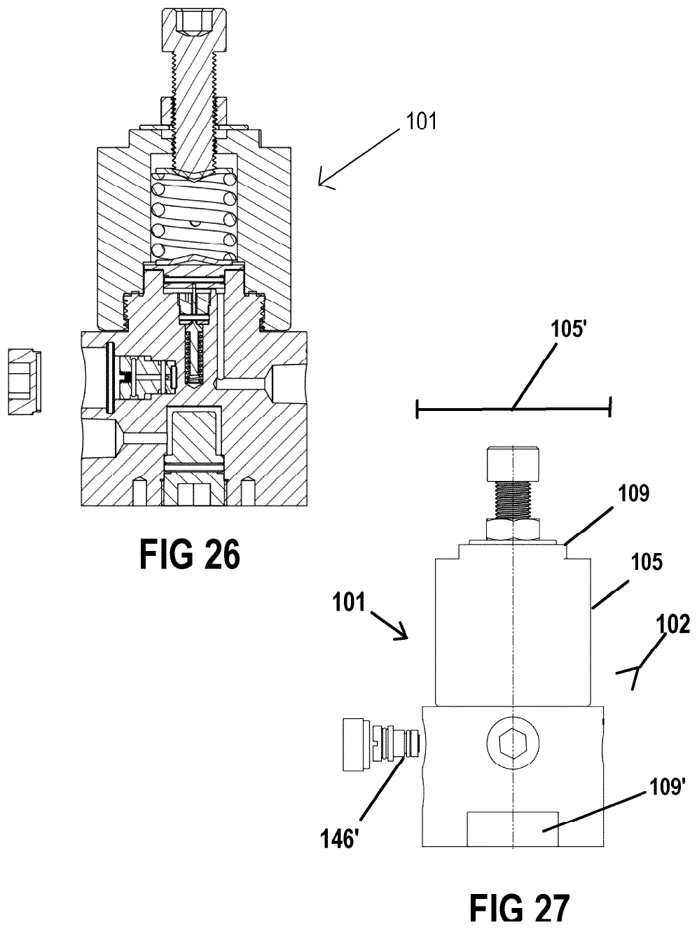

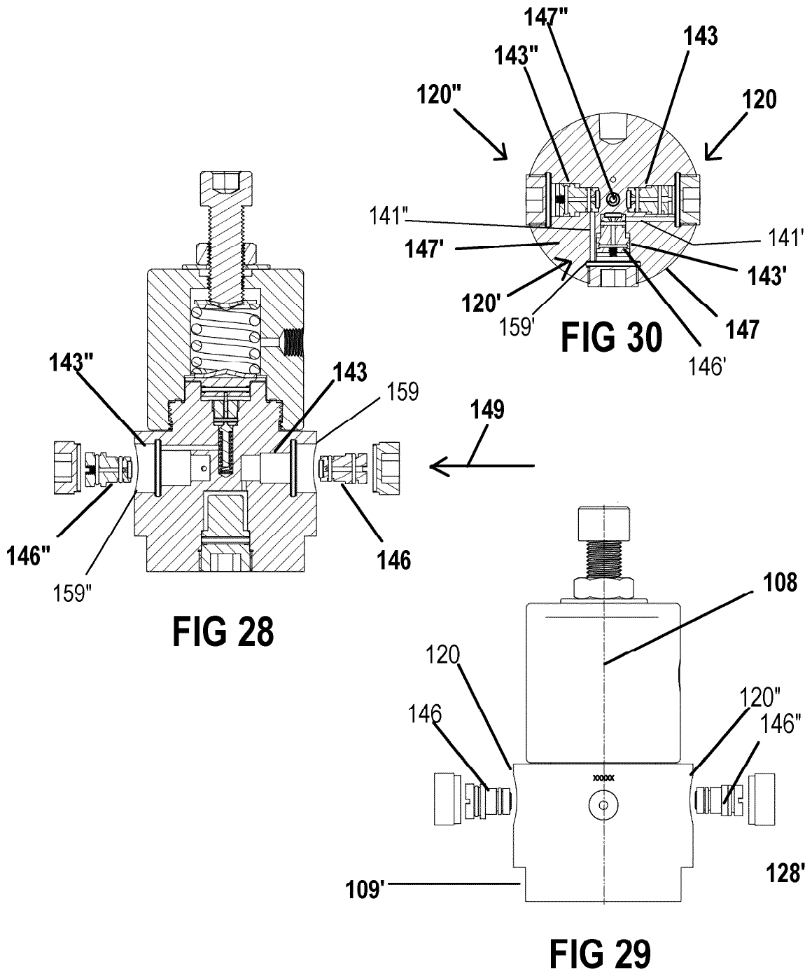

Continuing with the FIGS. 22-30, a third embodiment of the invention of the present application, comprises a fluid conditioning device 101 in the present example, in the form of a multi-stage regulator, sharing some features similar to the prior embodiments associated with FIGS. 2-8A, but also having differences, such as lacking a built-in vaporizer and having a pre-treatment stage capacity. Like the first and second embodiments of the invention discussed herein, the present embodiment is configured to provide first, second, and third fluid conditioning stages having serial flow therethrough in the unique radial design as described herein, in the form of a series of modular conditioning chambers or receivers, (also referenced as “piston chambers”) formed to receive “drop-in” modular conditioning components (in the present example, regulator components for pressure reduction, although other apparatus for other functions are also available), each of which are independently accessible via respective access ports or openings formed at the outer surface of the device body.

The present invention also provides optional pre-heat or pre-conditioning capacity, as well as a final stage comprising an adjustable regulator, the present exemplary embodiment of the device illustrating a multi-stage regulator configured to provide stepped-pressure reductions so as to avoid or diminish Joule-Thomson effect condensation of wet gas, as well as the method of using same, as will be discussed further herein.

It is noted that, while the present illustrated example of the present invention teaches three radially situated modular conditioning component chambers (also referenced as “piston chambers”) to receive “drop in” fluid conditioning components to provide three stages of stepped fluid conditioning, the actual number of stages can vary depending on the application and circumstances of use.

As used herein, a “drop-in” modular conditioning component or “piston” refers to unitary or integrated element(s) having a generally uniform outer configuration to facilitate insertion into a receiver or chamber (referenced as “piston chamber” or “component chamber”) formed in the body of the device to receive such a component's outer or exterior configuration, and in the present (non-limiting) example, being of a generally “piston-shaped” or cylindrical exterior form. As used in this context, the term “piston” refers solely in an exemplary context to a general geometric configuration—such as a generally cylindrical or elongated profile-and is not intended to imply or require the presence or function of an actual piston or reciprocating element, unless expressly stated otherwise. Further, the modular conditioning component or “piston” as can be utilized in the present invention can exist in other configurations including a non-radial, rectilinear form, for “drop in” to a chamber or receiver formed to receive same.

The “drop-in” feature means that the modular conditioning component (shown in the form of a single-stage regulator) can be added to or removed from the piston chamber without the need for permanent attachment, specialized tools, or significant assembly or disassembly whether in the field or lab, thereby enabling an easier implemented, less intensive modular installation, replacement, repair, or reconfiguration, etc., anywhere, including in the field and other less than ideal conditions outside of a lab or service facility.

The modular conditioning component is not intended to be restrictive as to its functionality or purpose (even though the exemplary embodiment comprises a regulator function), and it is intended that the component can vary depending on the application. Accordingly modular conditioning components comprise devices performing any of a variety of functions as described throughout the present application, including but not limited to, fluid regulation, flow reduction, filtration, separation, monitoring, or temperature adjustment/moderation/control. The term “piston-shaped” is understood to include both generally cylindrical forms as well as other elongated profiles suitable for cooperative engagement with the walls of a piston chamber. The term “piston chamber” is likewise not intended to be limiting as to operation, and should be construed as a configurational description of a component dimensioned and formed to receive and operatively interact with the drop-in component configured to receive same.

Continuing with the FIGS. 22-33, the device 101 of the present invention comprises a body 102 having an overall length 103 defining first 103′ and second 103″ ends and forming a longitudinal axis 108, the body further comprising threadingly 110 connected, first or upper 104 and second or lower 104′ sections comprising an upper adjustable regulator section 106, a lower base 107 and a plurality of modular conditioning component or “piston” chambers situated therebetween.

The piston chambers (shown as first 143, second 143′ and third 143″ piston chambers) are shown serially engaged via respective flow passages, in a radial arrangement in the lower 104′ section of the base, the piston chambers (143, 143′, 143″) formed to receive one or more modular conditioning components (as will be more fully discussed herein), the overall (assembled) device 101 shown as having a cylindrical outer surface 105 defining an outer diameter 105′ with wrench flats 109, 109′ formed in the upper 104 and lower 104′ sections, respectively, to facilitate engagement (and tightening or loosening) via an open-ended wrench or the like.

A fluid inlet 128, which can comprise, for example, an NPT inlet connection 128′, is provided at about the second end 103′ of the lower section 104′ of the body 102 to receive a flow of fluid 133 (wet gas or the like) therein.

The base 107 at the second end 103′ of the body 102 has formed therein a flow or pre-treatment chamber 112 having an inner wall 112′ forming an inner diameter 112″ a threaded 115′ opening 115 formed to receive a cover 116 with O-ring 116′ or other seal to selectively provide a access port to chamber 112. The pre-treatment chamber 112 can be sized and configured to receive modular conditioning components, for example those similar to the aforementioned piston chambers 143, 143′, 143″, so as to receive a drop-in modular conditioning component such as described herein to provide optional a pre-conditioning stage for fluid passing therethrough before reaching said serially-linked piston chambers 143, 143′, 143″.

Alternatively, and as shown in the present illustrated example, instead of adding a drop-in modular conditioning component, the pre-treatment chamber 112 as shown in the figures has situated therein a plug 113 of heat conductive material such as stainless steel or the like, the plug 113 having dimensions sized to encompass much of the volume of said pre-treatment chamber 112, while providing a clearance 114 between the inner wall 112′ of the chamber and the outer wall 114′ of the plug 113, so as to provide a flow passageway 129 to facilitate the flow of fluid 133 from the inlet 128 therethrough, and where the base 107 and plug 113 are heated via a block heater or the like, so as to provide a heated chamber 112 and passageway 129, fluid 133 flowing therethrough is pre-heated as a pre-treatment stage in the present system, as will be further discussed infra.

It is noted the pre-treatment chamber 112 may also serve as simply a pass-through for fluid flowing therethrough, when pre-treatment is not required. In such an application, the plug 113 (if situated in chamber 112) may remain but not be heated, or may be removed with the pre-treatment chamber sealed to allow flow of fluid through an empty chamber 112. Alternatively, a conditioning or sensor component may be inserted in the chamber.

Flow passage 141 provides a passage of fluid from the pre-treatment chamber 112 (which may or may not pretreat the fluid flowing therethrough as discussed above and herein) to the first stage 120 of four serially-connected conditioning stages, stages 1-3 (120, 120′, 120″ respectively) shown situated in a radial positioning relative one another relative to a common center point 147″, each stage each comprising piston chambers 143, 143′, 143″ formed to receive drop-in, modular regulator components 146, 146′, 146″, respectively, the piston chambers 143, 143′, 143″ each having independent access ports or openings situated along the outer radial surface 147 of the cylindrical body 148 of the device, said piston chambers 143, 143′, 143″ situated in serial, spaced relationship relative to one another along a common plane 147′ and linked via passages 141′, 141″ from one to the other in series, respectively. As shown, said plane 147 is positioned perpendicular to said longitudinal axis 108 of the body 102 of the device 101 with the piston chambers 143, 143′, 143″ shown in lateral orientation towards the side of the body.

As used herein, the term “piston chamber” is not intended to be limited to a chamber formed specifically to receive a piston or a particular shape or functionality, and can be also referenced as a “modular conditioning component chamber” or the like. Further, “piston chamber” is intended to be construed broadly to encompass various shaped chambers, receivers or the like to contain various diverse components, including but not limited to receiving a modular conditioning component, pressure reduction component, a heat transfer component or other application as described in the present application, or no component at all, to function as a pass-through, for example. The use of the term is for convenience and clarity, and should not be interpreted as restricting the scope of the invention to any particular element, embodiment or configuration unless expressly stated otherwise.

Each modular conditioning component chamber or piston chamber 143, 143′, 143″ has a threaded opening 144, for receiving a threaded cover 145 formed to engage O-Ring 145′ or other seal to provide an access port to a sealed chamber 142, each said chamber sized to receive a drop-in modular conditioning component (or “piston”) in the form of a modular regulator component 146, 146′, 146″ therein respectively, with each said each component 146, 146′, 146″ inserted 149 or otherwise placed in to their respective piston chamber 143, 143′, 143″, and sealed therein via threadingly engaging a respective cover 145 with O-ring 145′ to the threaded opening 144 of each piston chamber. As discussed, in the present exemplary embodiment, each modular conditioning component comprises a regulator or pressure reducer, to provide staged pressure reduction of fluid flowing serially therethrough, so as to prevent Joule-Thompson condensation of wet gas (gas having entrained liquid therein), and provide pressure reduced wet gas for analytical testing or other application.

Applicant assignee's patents U.S. Pat. No. 10,690,570 or U.S. Pat. No. 8,220,479B1, the contents of which are incorporated herein via reference thereto, teaches examples of a modular single stage pressure ratio regulator (see for example 38 in FIG. 8b, which is from applicant's U.S. Pat. No. 10,690,570) illustrating the various components and operation which could be utilized in modular regulator component for the present application, although it is emphasized the item illustrated in FIG. 8 is provided to illustrate the general operational characteristics and elements of a device which could prove suitable for the present application, although the device would have to be configured and sized to fit the present application.

In the present example wherein there is provided staged pressure reduction via serial flow of wet gas through conditioning stages 120, 120′, 120″, a pressure reducing component in the form of a modular regulator or conditioning component in the present exemplary embodiment would utilize a reduction piston or the like configured to provide a fixed pressure reduction, selected from a collection similar sized of modular regulators configured to provide various pressure cuts which could be inserted into the modular conditioning chamber(s) a/k/a piston chamber(s) of the present device. Each modular regulator component of the present invention is selected to provide the desired pressure cut ratio for each stage. The configuration of the piston housing of a particular component may be customized to accommodate both the inside dimensions required to contain the required piston within the piston housing, as well as the outer dimensions required to fit within the chamber to which it to be inserted.

Continuing with the figures, each modular conditioning component in the present exemplary embodiment is provided in the form of drop-in modular regulator component 146, 146′, 146″ for the present application, and comprises a piston housing which is formed to receive pressure reducing piston provided to reduce the pressure of wet gas flowing therethrough, providing a pressure cut of the fluid flowing into the vaporizer to facilitate the flash vaporization thereof, providing vaporized gas, which can then be further conditioned downstream, i.e., flowing to pressure regulator (such as an adjustable regulator) as further discussed herein.

Each modular regulator component 146, 146′, 146″ is selected to provide a staged pressure drop or pressure cut of the fluid flowing therethrough, with multiple staged components in series, such as the present invention, to provide a more gradual pressure reduction, to diminish the excessive amount of cooling from taking place in single stage systems (many times the Joule-Thomson (JT) cooling that takes place when a gas is reduced in pressure).

A further advantage of the present “drop-in” modular component feature is that it allows ready customization of the desired pressure drop to facilitate the desired vaporization or other conditioning required, by choosing the appropriate specification pressure reducer component apparatus, which can offer different pressure reduction characteristics while maintaining similar exterior dimensions/configuration, but with different pressure reduction characteristics, allowing the user to choose the desired pressure reduction characteristic for the particular installation, and insert or “drop in” the appropriate pressure reducer component into the pressure reduction component receiver (also described as piston chamber or modular conditioning component receiver of the unit.

Of the first 120, second 120″, third 120″ and fourth regulator stages, stages 1-3 comprise “drop in” modular conditioning components (in this case, in the form of regulator components) and the fourth being a manually adjustable regulator 150, thereby providing customizable, stepped-pressure reductions so as to avoid or diminish Joule-Thomson effect cooling and associated condensation of the vaporized sample flowing therethrough, the figure further illustrating utilization of the PTC heater cartridge of the present invention shown provided therein, as well as the passage from the vaporizer to the first regulator stage, and passage from the third regulator stage to the fourth regulator stage.

The radial design configuration of stage 1-3 modular regulator components 146, 146, 146″, respectively, of the present embodiment of the invention are situated in a unique radial configuration along the radial plane 147′ just upstream the pre-treatment chamber 112 (where utilized) provide an efficiency of space, while exploiting exploit any pre-heat characteristics where a heater is used, such as a block heater 154 mounted to the base 107 of the unit to heat said unit, providing heat to said pre-treatment chamber 112 and any heat conducting plug 113 situated therein to provide a heated pretreatment chamber flow passageway 129 resulting in heated, pre-treated fluid flowing therefrom. Further the relatively close physical proximity of the treatment stages 120, 120, 120″ can result in heating of same when the body 102 is formed of heat conducting material such as stainless steel, exploiting the thermal conductivity of said body 102 to transfer heat from the vaporizer area to the conditioning stages 120, 120′, 120″.

Unlike prior art systems, the unique radial design of the present invention, with the associated first, second and third piston chambers 143, 143′, 143″ respectively, each having an independent access means via an access port 159, 159′, 159″, each comprising a threaded opening 144 with removeable cover 145, provides easy, individualized access to each chamber and associated modular conditioning component or piston in a relatively small overall footprint, to allow for individual changes and/or maintenance as required even in the field, as opposed to having to require partial or full removal and disassembly of the unit to change or repair even an individual regulator stage, or replace O-ring seals as required on an individual basis as in various prior art systems.

For example, even in the field, one can easily access the drop-in modular conditioning component within a chamber via adjacent access port, perform the desired operation (replace, reconfigure, remove, etc.) then close the access port via a threaded cover 145 with seal, thereby providing for relatively easy reconfiguration, maintenance, or even removal of a particular stage (when a reduction stage is not required) or another sample conditioning or sensing component may be added in its place.

Following passage through the third piston chamber 143 and associated regulator component 146″ (when used), the reduced fluid then flows via passage 139 to the final, adjustable regulator 150 for a final pressure reduction or regulation, said adjustable regulator 150 having the same components and operation as the adjustable regulator of the single-stage, first embodiment of the invention shown in FIGS. 2-3, above, with the description of same is incorporated herein via reference thereto.

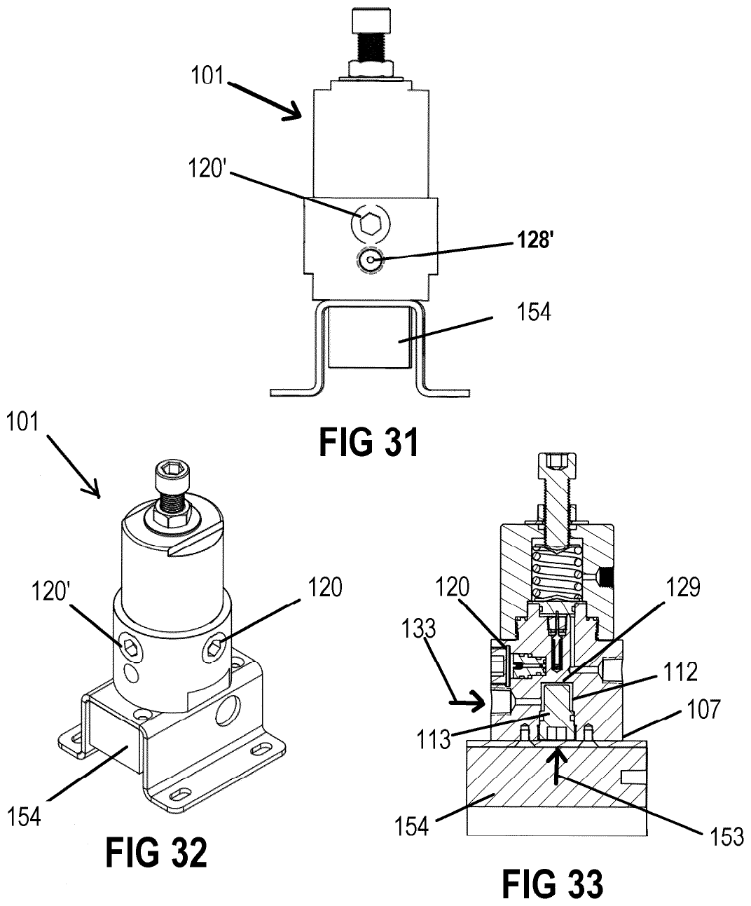

After passage through the final stage (the adjustable regulator 150), the pressure reduced fluid flows through passage 151, which passage directs the fluid back to the lower section 104 of the body so that where said body is heated such as via a heater block (FIGS. 31-33 as will be discussed herein) post regulator heat exchange of the fluid may be provided before it flows 152 out of the device via outlet 128″, which is situated along the same radial plane 147′ as the stage 1-3 regulator components 146, 146′, 146″, and associated conditioning stages 120, 120′, 120″, respectively.

With the mounting of a block heater 154 (for example, NTC or self-regulating) to the base 107 (as shown in FIGS. 31-33), and the application of heat 153 therefrom, the plug 113, formed of conductive material in the pre-treatment chamber, said heat is thereby conducted through the base 107 and provides heat (when said block heater 154 is attached and energized) via thermal conduction to said pre-treatment chamber 112 and associated flow passageway 129, as well as any said fluid flowing 133 therethrough, thereby providing preheated fluid as well as (to a lesser degree due to dissipation) heat to conditioning stages 120, 120′, 120″ and post regulation heat exchange passage 151, as discussed supra.

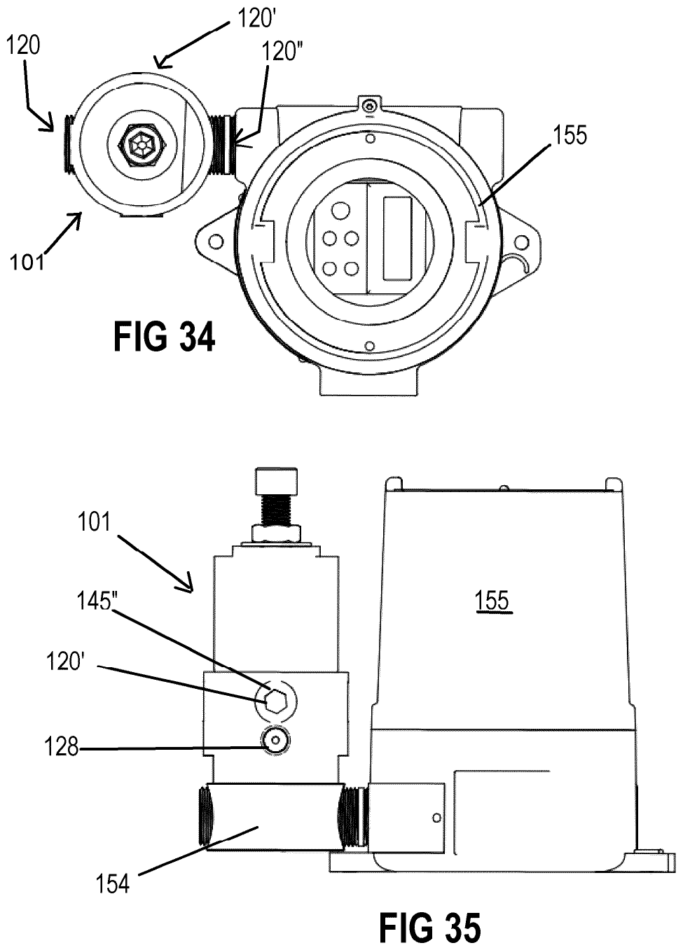

FIGS. 34-35 illustrate an exemplary installation of the device 101 of FIGS. 22-30 with heat block 154 install as shown in FIGS. 31-33, the device with heat block mounted for use with an NTC controller 155 in a sampling application. Notice independent accessibility of each of the first 120, second 120′ and third 120″ fluid conditioning stages wherein one simply depressurize the system, remove the threaded cover for the desired stage to be serviced, repaired or reconfigured (for example, for reconfiguring or servicing the second stage, threaded cover 145′ can be removed to access second stage 120′, at which point a modular regulator component (such as 146′ shown in FIG. 30) can be removed for replacement, reconfiguration, or simply removal of that stage, then, upon completion, the threaded cover 145′ is re-installed to seal the stage and the system is ready for re-pressurization/reuse.

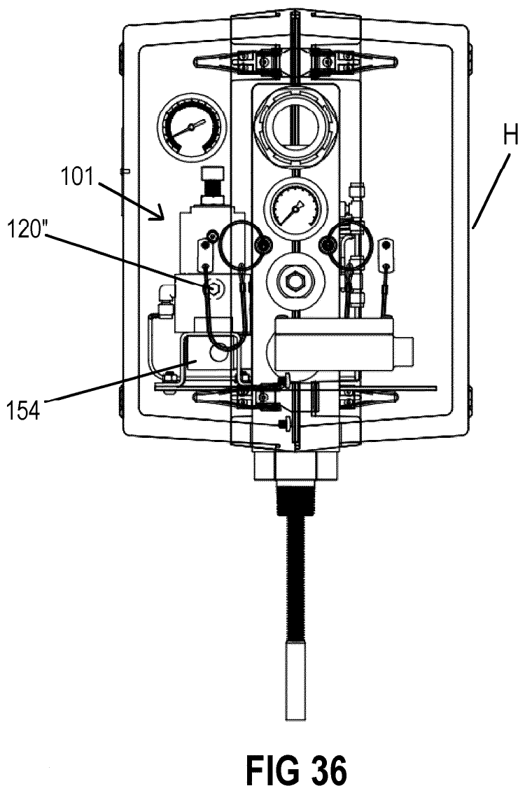

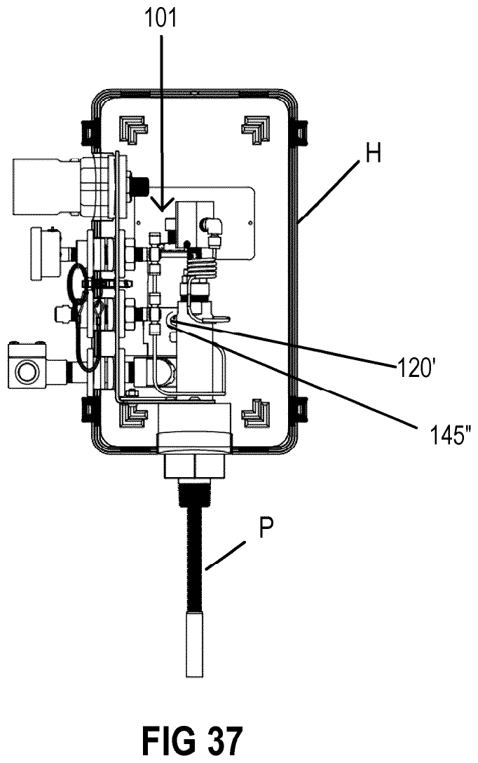

FIGS. 36-37 show an exemplary installation of the device 101 with heater block 154 with a capillary probe P or the like and insulated housing, the install configured to be mounted, for example, to a pipeline. Notice easy independent accessibility of the stages (covers for accessing stages 120′, 120″ shown in FIGS. 36 and 37, respectively). For reconfiguration of for example the second stage 120′ of device 101, the insulated housing H would be opened or removed, leaving the user able to access each stage independently in the device with removal of device 101 or disassembly of same. For example, for reconfiguring or servicing the second stage 120′ of the device 101, threaded cover 145′ is removed to access the underlying second conditioning component chamber, or piston chamber (146′ in FIG. 30) chamber second stage 120′, at which point a modular regulator component (such as 146′ shown in FIG. 30) can be removed for replacement, reconfiguration, or simply removal of that stage, then, upon completion, the threaded cover 145′ is re-installed to seal the stage and the system is ready for re-pressurization/reuse.

While the present invention illustrates a radially-configured, multi-stage, modular conditioning device each staged formed to receive drop-in modular conditioning components which are easily and independently accessible for each stage, the stages shown in a relatively compact, space saving radial configuration, it is emphasized that the radial configuration is not intended to be limiting, and can be provided in other configurations with similar efficiencies, as will be detailed further herein.

### Regulator With Stacked, Multi-Stage, Serial Flow

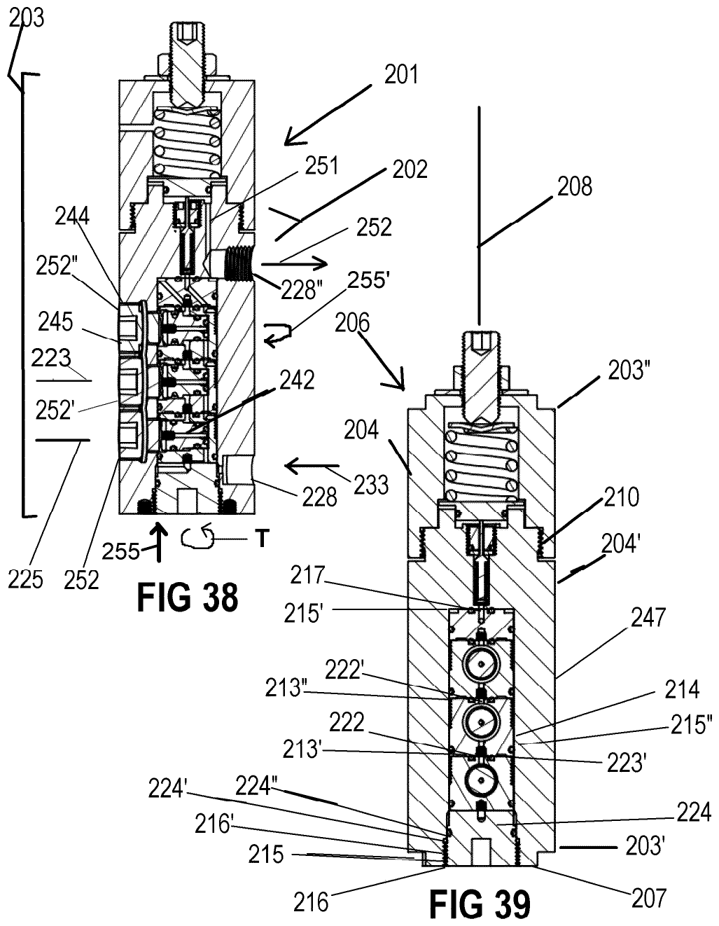

FIGS. 38-43 illustrate a fourth embodiment of the multi-stage regulator of the inventions of FIGS. 2-8A and FIGS. 22-30, illustrating a body having provided therein in first, second and third situated conditioning stages (illustrated in the form of regulator stages) in a linear “stacked” configuration (as opposed to the “radial” configuration illustrated in the previous embodiments taught herein).

As with the previous embodiments, each stage in the present embodiment is configured to facilitate independent accessibility of the modular conditioning component (in the present example, a single-stage regulator) situated therein via adjacent access port or opening formed in the body, said stages configured for serial flow therethrough to a fourth, adjustable regulator stage, so as to provide four stages. The present exemplary application, as in the previous embodiments, is illustrated in the form of a multi-stage regulator configured to provide stepped-pressure reductions so as to avoid or diminish Joule-Thomson effect condensation of a wet gas sample, including passage to the first regulator stage, and passage from the third regulator stage to the fourth regulator stage, although this application is for illustrative purposes only and is not intended to be limiting.

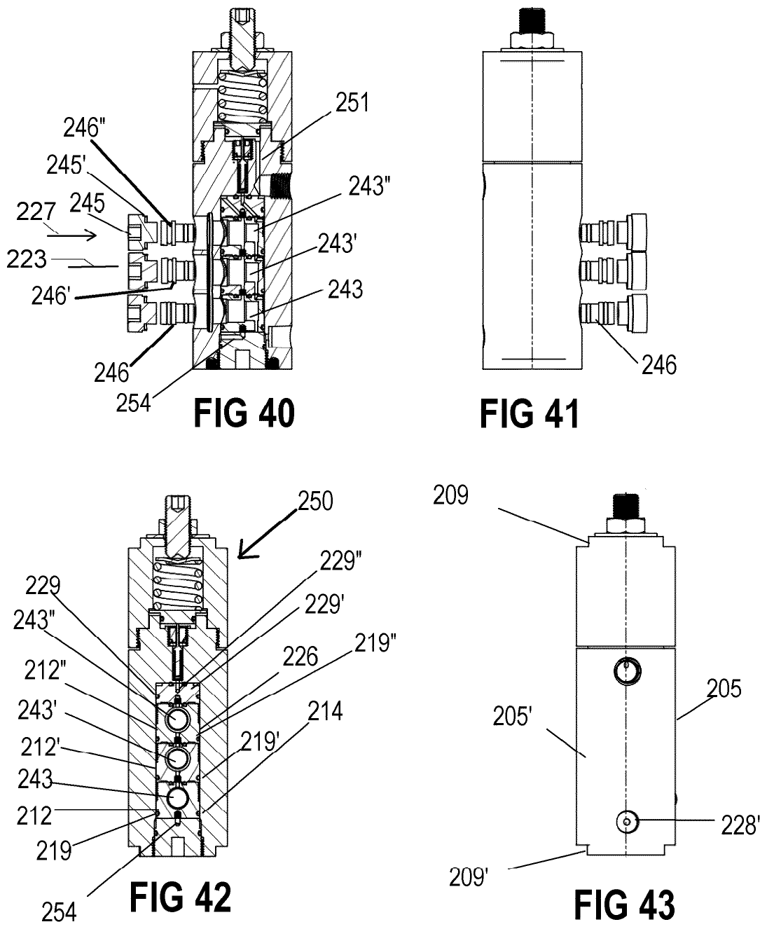

FIGS. 40-42 provided details as to the threaded covers and modular conditioning components (discussed in the form of single-stage regulator components) showing the components removed from the stacked modular component receivers and associated access ports or openings for said first, second and third and third regulator stages, respectively.

FIG. 42 is a cross-sectional, third side view of the multi-stage regulator of FIG. 39, with the modular conditioning components removed, illustrating stacked first, second and third modular component receivers formed to receive said first, second and third modular conditioning components as referenced herein.

FIG. 43 is an opposing, third fourth side vide of the multi-stage regulator of FIG. 42, illustrating the outer body and upper adjustable regulator (forming the fourth stage in the present embodiment), upper flow outlet port and lower flow inlet port.

Continuing with FIGS. 38-43, the device 201 of the present invention comprises an elongated body 202 having an overall length 203 defining first 203′ and second 203″ ends, the body further comprising threadingly 210 connected, first or upper 204 and second or lower 204′ sections comprising an adjustable regulator section 206, a base 207 and a plurality of stackable chamber components for receiving modular regulator components situated therebetween, respectively, as further described herein.

The body 202 of device 201 has formed therein a longitudinal receiver 214 having first 215 and second 215′ ends and an inner sidewall 215″, the first end 215 of longitudinal receiver comprising a threaded 216′ opening 216 in the base 207 formed to receive a threaded 224′ insert 224 with o-ring 224″ or the like to provide a sealed connection, the second end 215′ terminating into a flow passage 217 leading to adjustable regulator 206.

The longitudinal receiver 214 is formed to receive, in stacked arrangement from the base, first 212, second 212′, and third 212″ stackable chamber components, respectively, each said stackable chamber components 212, 212′, 212″ having formed therein a modular component receivers or piston chamber 243, 243′, 243″, respectively, said receiver or chamber(s) positioned in said longitudinal receiver so as to be situated in a lateral 225 orientation relative to the longitudinal axis 208 of the body, when installed in said receiver 214.

Each said chamber component is situated in stacked form upon the other in said longitudinal receiver to facilitate insertion or removal therefrom as required. Upon placing or inserting 255 each chamber component 212, 212′, 212″ into the longitudinal receiver 214, it is important that the piston chamber 243, 243′, 243″ formed in each said chamber component be in alignment 223 with the respective access port 252, 252′, 253″. This can be accomplished by rotating 255′ or axially adjusting each chamber component 212, 212′, 212″ respectively to the extent required to ensure that each respective piston chamber 243, 243′, 243″, is positioned to be in alignment 223 with its respective, adjacent access port 252, 252′, 252″, to ensure ready exterior access on demand to the respective piston chamber, and any modular conditioning component situated therein, via said respective access ports, the respective chamber components slidingly situated in stacked formation within the longitudinal receiver 214 and affixed in position, such as via orientation means, as further discussed herein, or manual alignment, and fixing said chamber components in place via tightening T insert 224, or other means, as discussed herein.

Each chamber component 212, 212′, 212″ has situated thereabout an o-ring 213, 213′, 213″ or similar sealing means to provide a fluid seal between each component to the inner sidewall 215″ of said longitudinal receiver 214. In addition, each chamber component 212, 212′, 212″ has an o-ring 219, 219′, 219″ or similar seal provided between the stacked components, situated to envelope a fluid passage 222, 222′ at opposing ends 213′, 213″ of said chamber components, and which are in alignment 223′ upon stacking of same, for serial communication therethrough.

Piston chambers 243, 243′, 243″ formed in each chamber component 212, 212′, 212″, respectively are formed to receive one or more modular conditioning components (as will be more fully discussed herein), the overall (assembled) device 201 of the present exemplary embodiment shown as having a cylindrical outer surface 205 defining an outer diameter 205′ with wrench flats 209, 209′ formed in the upper 204 and lower 204′ sections, respectively, to facilitate engagement (and tightening or loosening) via an open-ended wrench or the like.

Each access port 252, 252′, 252″ formed in the lower section 204′ of said body 202 is threaded to provide a threaded opening 244, for receiving a threaded cover 245 formed to engage O-Ring 245′ or other seal to provide a sealed chamber 242, each said chamber associated with said piston chambers, so as to receive a drop-in modular conditioning component (or “piston”) in the form of a modular regulator component 246, 246′, 246″ therein respectively, with each said each component 246, 246′, 246″ inserted 249 or otherwise placed in to their respective piston chamber 243, 243′, 243″, and sealed therein via threadingly engaging 227 a respective cover 245 with O-ring 245′ to the threaded opening 244 of each the respective access port 252, 252′, 252″. While the present example shows the threaded cover 245 with o-ring 245′ as separate and distinct from the modular conditioning component (shown as modular regulator components 246, 246, 246″) it is noted that alternatively the threaded cover 245 can be formed integrally with the modular regulator component to provide a one-piece unit, and thereby simplify removal and replacement of said modular regulator component(s).

As discussed, in the present exemplary embodiment, each modular conditioning component comprises a regulator or pressure reducer, to provide staged pressure reduction of fluid flowing serially therethrough, so as to prevent Joule-Thompson condensation of wet gas (gas having entrained liquid therein), and provide pressure reduced wet gas for analytical testing or other application.

Continuing with the figures, a fluid inlet 228, which can comprise, for example, an NPT inlet connection 228′, is provided at about the first end 203′ of the lower section 204′ of the body 202 to receive a flow of fluid 233 (wet gas or the like) therein to facilitate flow via passage 254 to the first chamber component 212 and associated modular conditioning component 246, where fluid therethrough is conditioned then flows to the second, third and fourth conditioning stages provided by the respective second and third modular conditioning components 246′, 246″, and adjustable regulator 250, as will be more fully discussed herein.

Unlike prior art systems, the unique modular features of the present invention, with the associated first, second and third piston chambers 243, 243′, 243″ respectively oriented n orientation to provide independent access means via a respective access port 252, 252′, 252″ for engagement with removeable cover 245, provides easy, individualized access to each chamber and associated modular conditioning component or piston, to allow for individual changes and/or maintenance as required, as opposed to having to require disassembly of the entire unit to change or repair an individual regulator stage, or replace O-ring seals as required on an individual basis as in various prior art systems.

In addition, one can simply access a drop-in modular conditioning component within the chamber, perform the desired operation (replace, reconfigure, service, remove, etc.) then by removing the treaded cover 145, then replace to seal the unit in place.

Following passage through the third piston chamber 243″ and associated modular conditioning component, in this case, regulator component 246″ (when used), the reduced fluid (having passed through the conditioning components) then flows through a sealed 229 (via o-ring) spacer component 229′ via central flow through passage 229″ to an adjustable regulator 250 for a final pressure reduction or regulation, said adjustable regulator 250 having the same components and operation as the adjustable regulator of the single-stage, first embodiment of the invention shown in FIGS. 2-3, above, with the description of same is incorporated herein via reference thereto.

After passage through the final stage (the adjustable regulator 250), the pressure reduced fluid flows through passage 251 before it flows 252 out of the device via outlet 228″.

As previously noted, the device of FIG. 20 illustrates applicant assignee's prior art GENIE brand JTR multi-stage Heated Regulator, described in U.S. Pat. Nos. 8,220,479, 8,616,228, and 9,588,024, the contents of which are incorporated herein by reference thereto, which like the present invention, of provides a system of stacked modular conditioning components, although the present invention of FIGS. 38-43 provides a vast improvement over this prior reference in a much less complicated, less costly to maintain and reconfigure (even in the field) when compared to the aforementioned system as, unlike these prior patented devices, provides easy independent access to each stage of the device for maintenance, repair or reconfiguration.

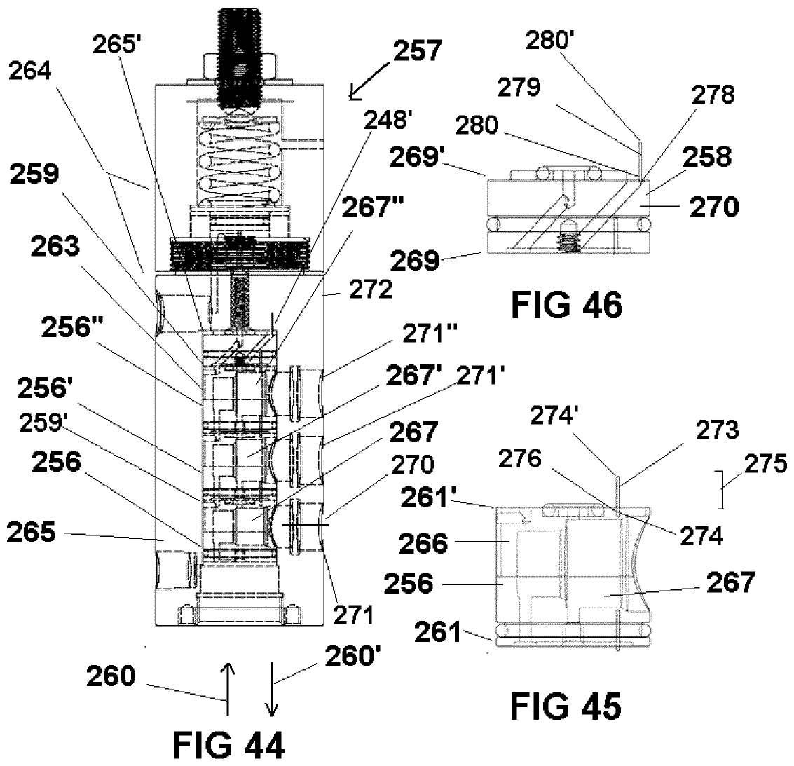

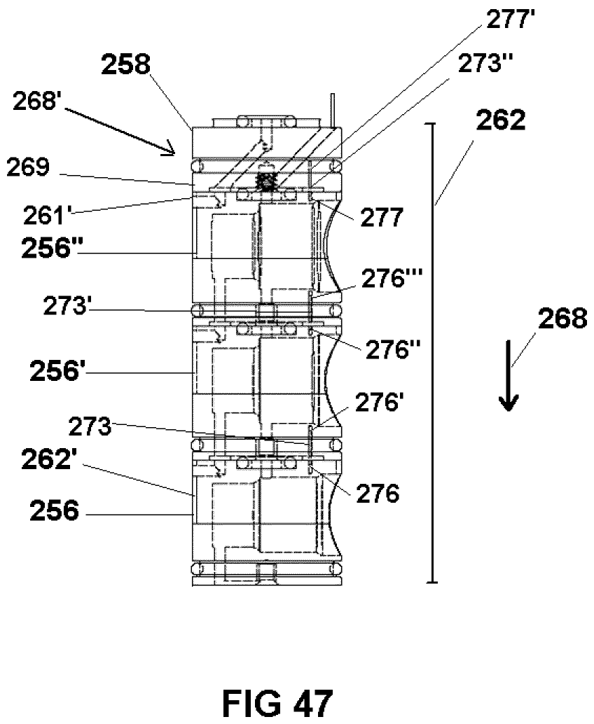

Lastly, FIGS. 44-47 illustrates an alternative embodiment 257 of the invention of FIGS. 38-43, wherein, as with the previous embodiment, each modular chamber component 256, 256′, 256′ (also referenced as “chamber component”) is stacked 268 upon the other, with a spacer block 258 situated at the top to form a stack 268′, the stack having a length and 262 and sidewalls 262′ dimensioned to allow insertion 260 into and sliding 263 engagement with the inner sidewall 259′ of the longitudinal receiver 259 of the body 265, as well as removal 260′ therefrom as required.

As shown in the present embodiment, each modular chamber component 256, 256′, 256″ has a bottom or first end 261 and top or second end 261′, with each said modular chamber component 256, 256′, 256″ having a side wall 266 having modular conditioning component receivers 267, 267′, 267″ (also referenced as “piston chamber” or “piston chambers”) formed therein, respectively. Further, spacer 258 has sidewall 270, a bottom or first 269 end and top or second 269′ end.

In order to facilitate proper orientation of the modular conditioning components 256, 256′, 256″ and ensure they are properly situated in longitudinal receiver 259 so that said respective modular conditioning component receivers 267, 267′, 267″ are in alignment 270 with respect to access ports 271, 271′, 271″ formed in said body 264, respectively, so as to provide ready access via the outer surface 272 of said body 264, each of said modular chamber components 256, 256′, 256″ are formed to receive and interconnect in stacked form via alignment pins 273, 273′, 273″, respectively, each said pin having a length 274 and first 274 and second 274′ ends.

To facilitate uniform alignment of the modular chamber component receivers 267, 267, 267″ of modular chamber component 256, 256′, 256″ respectively in stacked form, alignment pins are provided between the stacked 268 components to fix said components in position relative to one another and facilitate alignment of same.

Particularly, alignment pin 273 is provided to engage stacked modular chamber components 256 and 256′ via pin sockets 276, 276′ formed in said modular chamber components, respectively, while alignment pin 273′ is situated between stacked modular chamber components 256′ and 256″ via pin sockets 276″, 276″, respectively, formed in said modular chamber components. Further, alignment pin 273″ is provided and situated between the top 261′ of modular chamber component 256″ and bottom 269 of spacer block 258 via pin sockets 277, 277′ formed therein to engage same, respectively, the interconnected modular chamber components 256, 256′ 256″ and spacer 258 via said alignment pins 273, 273′,273″ forming an interconnected stack 268′, with modular chamber components 256, 256′, 256″ positioned in uniform, fixed alignment relative one another. Further, the top 269 of spacer block 258 has emanating therefrom alignment pin 279, with its first end 280, engaging pin socket 278 in top 269′ of spacer block 258, and the second end 280′ engaging pin socket 248′ at the second end 265′ of longitudinal receiver 259.

Said alignment pin 279 at the top of the stack 268′ is positioned to engage the second end 265′ of the longitudinal receiver 259 to ensure alignment of said stacked modular chamber components such that said respective modular conditioning component receivers 267, 267′, 267″ in said modular chamber components are in alignment 270 with respective access ports 271, 271′, 271″ of said body 24 when the stack 268′ is placed into longitudinal receiver 259 of said body, so the second end 280′ of said alignment pin 279 is inserted into said pin socket 248′ formed in said body 264 at said second end 265′ of said longitudinal receiver 259.

While the above embodiment illustrates as an example the use of pins to secure the modular chamber components in a fixed, uniform position relative to one another to ensure alignment of the modular component receivers with respective access ports as detailed above, further alternative means could be used to ensure said alignment. For example, a modular chamber component comprising a cylindrical sidewall configuration could include a rib formed to engage a groove in the sidewall of a cylindrical longitudinal receiver, the groove and rib positioned to ensure alignment of each piston chamber with its respective access port formed in the outer surface of the body of the device, to fix said cylindrical sidewall in a fixed position in said cylindrical longitudinal receiver, while allowing said modular chamber component to be slidingly positioned within said cylindrical longitudinal receiver.

### EXEMPLARY SPECIFICATIONS

### Exemplary Regulator Spec for Single Stage Vaporizing Regulator

Upper section: 2.230″ OD, length 2.2″
Lower section: 2.600″ OD, length 2.0″
Inlet: ¼″ 18 NPT, Outlet ¼″ 18 NPT

### Exemplary specs for each of four stage JT staged vaporizing regulator:

Upper section: 2.230″ OD, Length 2.2″
Lower section: 2.6″ OD, Length 2.0″
Inlet: ¼″ 18 NPT, Outlet ¼″ 18 NPT
Piston chamber ID: 0.370″, depth: 0.641″

### General Exemplary Specification

Heater Cartridge connection: ½″ NPT conduit connection
Conduit adapter connection: ½″ NPT conduit connection to 360-degree swivel connection
Exemplary temp range for heater: NEC Class I Div 1 Group D with a temperature classification of T3 200 degrees C. (392 F).
Exemplary “wet gas” characteristics: Natural Gas with less than 2% entrained liquid
Exemplary fluid flow rate/range input/output: Typical analytical flow rates of approximately one to three Standard Liters per Minute of vapor with an inlet pressure of typically less than 4,000 PSIG and a regulated outlet pressure of typically less than 100 PSIG. Some gas chromatographs typically only need 50 cc/min vapor flow rate at 25 to 50 PSIG, but emerging optical analyzers including Tunable Diode Lasers require up to three L/min vapor flow rate at only 10 PSIG (low pressure due to glass optical analyzer cells).

### RECITATION OF ELEMENTS

D Phase diagram
R,′ prior art single, 4 stage regulator
T Tighten
V prior art genie vaporizer
P capillary probe
H insulated housing
1 SS vaporizing regulator
2 vaporizer body
3,′,″ length, first, second ends
4,′ upper, lower sections
5,′ generally cylindrical configuration, OD
6 regulator section
7 vaporizer section
8,′,″ conduit adapter section, cylindrical plug, socket
9,′ wrench flats
10,′,″ threadingly connected
11,′ HC Base, threaded OD
12 heater cartridge section
13,′ Vaporizer first, second ends
14,′ length, longitudinal axis
15,′ opening, threaded sidewalls
16 receiver
17,′ ID, depth
18 end distal opening 15
19,′,″ HC Thermal conductive sleeve or jacket (AL), groove, O-rings
21 receive
22,′ conductor sleeve OD, length
23 base wrench flat
24,′,″ vaporization chamber, length, vaporization chamber (2nd embodiment)
25 space
26 conductor sleeve outer surface
27 mesh
28,′ fluid inlet, outlet
29 fluid flow
30 clearance between distal tip of conductor sleeve and distal end of receiver
31,′ post heat passage/outflow,
32 pivotal rotation
33,′,″ inlet flow, vaporized sample pre regulation, flow from regulator,
34 post regulation heat exchange passage
35,′ adjustable regulator, spring biased seat2
36,′ piston, adjustment mechanism, helical compression spring
37 prior art systems
38 modular single-stage regulator
39 passage between third regulator and final adjustable regulator
40 Multi-Stage Vaporizing regulator
41 post heat passage
42,′ inlet, outlet
43,′,″ first, second, third piston chambers
44 threaded opening or access port
45 threaded cover with O-ring or other seal
46, ′,″ modular regulator component
47,′ outer radial surface, radial plane
48 cylindrical body
49 inserted
50 adjustable regulator
51 post regulation heat exchange passage
52,′ vaporized, pressure reduced fluid
53,′ PTC heater cartridge, power connector
54 NTC heater cartridge
55,′,″ thermal cutoff, temp sensor, heater cartridge power
56 insulated enclosure
57 available space
58 center point
60 capillary flow path
61 sampling probe
62 vaporizer regulator
63 wet gas
101 third embodiment fluid conditioning device/multi-stage regulator device
102 body
103,′,″ length, first, second ends
104,′ upper, lower sections
105,′ generally cylindrical configuration, OD
106 adjustable regulator section
107 base
108 longitudinal axis
109,′ wrench flats
110,′,″ threadingly connected
111,′ HC Base, threaded OD
112 pre-treatment chamber, inner walls, ID
113 plug
114,′ clearance, outer wall plug
115 PT chamber opening, threaded
116 cover, O-ring
120,′,″ first, second and third, radially situated conditioning stages
123 base wrench flat
126 conductor sleeve outer surface
128,′,″ fluid inlet, NPT connection, outlet
129 pretreatment chamber flow passageway
131,′ post staged regulation passage/outflow (before adjustable regulator),
132 pivotal rotation
133,′,″ inlet flow, sample pre adjustable regulation, flow from regulator,
134 post regulation heat exchange passage
135,′ adjustable regulator, spring biased seat2
136,′ piston, adjustment mechanism, helical compression spring
137 prior art systems
138 modular single-stage regulator, drop-in component
139 passage between third regulator and final adjustable regulator
140 Multi-Stage regulator
141 flow passage
142 sealed chamber
143,′,″ first, second, third modular conditioning component chambers, or piston chambers
144 threaded opening or access port
145,′,″ threaded cover with O-ring or other seal
146, ′,″ modular regulator component
147,′,″ outer radial surface, radial plane, center point
148 cylindrical body
149 inserted
150 adjustable regulator
151 post regulation heat exchange passage
152 pressure reduced fluid
153 heat
154 NTC or self regulating block heater
155 NTC Controller
156 insulated enclosure
157 available space
158 capillary probe
159 access port
201 third embodiment fluid conditioning device/multi-stage regulator device
202 body
203,′,″ length, first, second ends
204,′ upper, lower sections
205,′ generally cylindrical configuration, OD
206 adjustable regulator section
207 base
208 longitudinal axis
209,′ wrench flats
210,′,″ threadingly connected
211,′ HC Base, threaded OD
212,′,″ chamber components
213,′,″ stacked, opposing ends
214 longitudinal receiver
215, ′,″ first and second ends, inner sidewall
216,′ opening at base, threaded
217 passage to adjustable regulator
218,′,″ O-rings about chamber component
219,′,″ O-rings between stacked chamber component
220,′,″,″ first end passage for chamber component
221,′,″ second end passage for chamber component
222,′ upper, lower passage in each chamber component 212 etc.
223,′ alignment
224,′,″ insert, threaded o-ring seals
225 lateral
226 profile
227 engaging
228,′,″ fluid inlet, NPT connection, outlet
229,′,″ sealed, spacer block, central passage
233 flow of fluid
242 sealed chamber
243,′,″ first, second, third modular conditioning component receivers, or piston chambers
244 threaded opening or access port
245,′,″ threaded cover with O-ring or other seal
246, ′,″ modular regulator component
247 outer radial surface
248 cylindrical body
249 inserted
250 adjustable regulator
251 outlet passage
252,′,″ access ports
254 insert flow passage
255,′ placing, rotating
256,′,″ modular chamber component, also referenced as chamber components
257 alternative embodiment
258 spacer block
259,′ longitudinal receiver, inner sidewall
260,′ insertion, removal
261,′ bottom, top modular chamber component
262,′ length, sidewalls
263 sliding engagement
264 body
265,′ first, second ends of longitudinal receiver
266 modular chamber component side wall profile
267,′,″ conditioning component receivers, also referenced as piston chambers
268,′ stacked, stack
269 bottom, top (spacer)
270 alignment
271,′,″ access ports
272 outer surface
273,′,″,″′ alignment pins
274,′ first second ends
275 length
276, ′, ″,′″ pin sockets
277,′ pin socket top mcc, spacer bottom
278,′ pin socket top of socket, body @ second end longitudinal receiver
279 alignment pin at top of stack
280,′ first, second ends top pin (spacer block)

The terminology used herein is for the purpose of describing particular embodiments only and is not intended to be limiting of the invention. As used in the specification and appended claims, singular forms “a,” “an,” and “the” include plural referents unless the context clearly dictates otherwise. It will be further understood that the terms “comprises,” “comprising,” “includes,” and/or “including,” specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms used should be interpreted as having a meaning consistent with the context in which they are used in the specification and claims. No term or phrase should be interpreted as being limited to a specifically described embodiment or example unless explicitly stated otherwise.

The invention embodiments herein described are done so in detail for exemplary purposes only, and may be subject to many different variations in design, structure, application and operation methodology. Thus, the detailed disclosures therein should be interpreted in an illustrative, exemplary manner, and not in a limited sense.

I claim:

1. A multi-stage fluid conditioning device comprising:
a body having first and second ends forming a length and a longitudinal axis, said body further comprising an outer surface,
a plurality of piston chambers situated in said body, each of said piston chambers linked in serial fashion to an adjacent piston chamber so as to provide a series of linked piston chambers comprising first and last serially-linked piston chambers, each piston chamber having associated therewith an access port to exterior said body;
an inlet comprising a first flow passage to said first serially-linked piston chamber, and a flow passage from said last serially-linked piston chamber;
each said piston chamber formed to receive a modular conditioning component via said respective access port, each said modular conditioning component selected from a collection of modular conditioning components having various fluid regulating characteristics, each said modular conditioning component formed for insertion into and containment via said respective piston chamber and associated access port;
whereby, upon inserting said modular components into said piston chambers and sealing said piston chambers via said respective access port, providing piston chambers having said selected modular conditioning components sealed therein respectively in a desired order, providing a series of linked modular conditioning components, and facilitating serial flow of fluid therethrough, so as to provide a staged conditioning of fluid flowing therethrough via said modular conditioning components.

2. The multi-stage fluid conditioning device of claim 1, wherein one or more of said modular conditioning components comprise single-stage regulators.

3. The multi-stage fluid conditioning device of claim 2 wherein there is provided a flow passage from said last serially-linked piston chamber to an adjustable regulator having an adjustment mechanism engaging said body, said adjustable regulator providing adjustably regulated flow to fluid flowing therethrough to a post regulator passage.

4. The multi-stage fluid conditioning device of claim 3, wherein said device body has a generally cylindrical configuration having an outer surface forming an outer diameter, and wherein said access ports of said serially linked piston chambers are situated along said outer surface of said body.

5. The multi-stage fluid conditioning device of claim 4, wherein said fluid comprises a gas having entrained liquid, and wherein said inlet is formed to receive a flow of sample fluid via a capillary flow path from a sampling probe.

6. The multi-stage fluid conditioning device of claim 4, wherein said piston chambers are situated along a common plane perpendicular to said longitudinal axis of said body in a radially situated configuration relative to one another.

7. The multi-stage fluid conditioning device of claim 6, wherein there is further provided an adjustable regulator downstream said series of linked conditioning components.

8. The multi-stage fluid conditioning device of claim 3, wherein said piston chambers are laterally oriented in relation to said longitudinal axis of said body, each said piston chamber situated in stacked configuration in said body, providing adjacent modular chamber components in sealed engagement, so as to facilitate serial flow from one to the other.

9. The multi-stage fluid conditioning device of claim 8, wherein each said modular conditioning component said piston chamber formed in a separate, respective modular chamber component.

10. The multi-stage fluid conditioning device of claim 9, wherein each said piston chamber formed in each said respective modular chamber component is oriented such that each said modular conditioning component situated therein is oriented toward said associated access port to facilitate independent access to same.

11. The multi-stage fluid conditioning device of claim 10, wherein said modular chamber components are engaged to one another in stacked, serial relationship via alignment pins to facilitate orientation of said modular conditioning components therein in relation to their respective said access ports.

12. The multi-stage fluid conditioning device of claim 11, wherein there is further provided an adjustable regulator downstream said series of linked conditioning components.

13. A method of conditioning a fluid, comprising the steps of:
a. providing multi-stage conditioning device comprising a body having an outer surface, a length, and a longitudinal axis, said body formed of thermally conductive material, said body further comprising a plurality of piston chambers, said piston chambers linked in serial fashion, providing a series of linked piston chambers comprising first and last serially-linked piston chambers, each of said piston chambers having provided therefore an access port associated with said outer surface of said body, said serially-linked piston chambers configured to provide an order of flow from one of said piston chambers to the other;
b. selecting from a collection of modular conditioning components having defined fluid conditioning characteristics to provide a predetermined order of components to facilitate staged conditioning of said wet gas;
c. placing said modular conditioning components in said predetermined order in said serially-linked piston chambers via said respective access ports, beginning with said first piston chamber, such that a modular conditioning component is placed in a single piston chamber in the desired order of flow, providing piston chambers having a modular conditioning component situated therein, providing a series of linked modular conditioning components;
d. sealing said access ports for said piston chambers;
e. flowing said wet gas fluid through said series of modular conditioning components situated in said serially-linked piston chambers; while
f. utilizing said modular conditioning components to provide stepped conditioning of said wet gas flowing therethrough.

14. The method of claim 13, wherein said fluid comprises wet gas, in step “b” said modular conditioning components comprise single-stage regulators linked in series, and in step “f” said series of linked single-stage regulators provide stepped regulation of said wet gas flowing therethrough so as to prevent Joule-Thomson effect condensation, providing JT regulated, pressure-reduced wet gas.

15. The method of claim 14, wherein there is further provided after step “f” the added steps of:
a. flowing said JT regulated fluid through a final adjustable regulator;
flowing said JT regulated fluid through a post regulation passage to a fluid outlet.

16. The method of claim 14, wherein in step “a”, there is provided the additional step a.(i) of situating said piston chambers in said body along a common plane perpendicular to said longitudinal axis of said body in a radially-situated configuration relative one another.

17. The method of claim 14, wherein in step “a”, there is provided the additional step of a.(i) situating said modular conditioning components in a stacked configuration in a lateral orientation relative to said longitudinal axis of said body of said device in sealed engagement, so as to facilitate serial flow from one to the other.

18. The method of claim 17, wherein said in step “a” there is provided the additional step of a.(ii) situating each said modular conditioning component in a separate, respective modular chamber component, and stacking said modular chamber components upon one another to form said stacked configuration, providing stacked modular chamber components.

19. The method of claim 18, wherein in step “a” there is provided the additional step of a.(iii) orienting each said modular chamber component comprising said stacked modular chamber components such that each said modular conditioning component situated therein is oriented toward said respective access port so as to facilitate independent access to same.

20. The method of claim 19, wherein in step “a”, there is provided the additional step of a.(iii) fixedly engaging said stacked modular chamber components to one another so as to fixedly position said modular chamber components so that each said respective modular conditioning component situated therein respectively is positioned adjacent to their respective said access ports.

21. The method of claim 20, wherein in step a.(iv) said modular chamber components are fixedly engaged via alignment pins situated between said stacked modular chamber components.

22. A multi-stage fluid conditioning device comprising:
a body having first and second ends forming a length and a longitudinal axis, said body further comprising an outer surface, said body further having formed therein a longitudinal receiver having first and second ends and an inner sidewall, said first end of said longitudinal receiver comprising an opening;
a plurality of chamber components, each having a piston chamber formed therein, said chamber components formed to be slidingly situated in said longitudinal receiver of said body in stacked fashion such that said piston chamber of each chamber component is situated in a lateral orientation relative to said body, each of said chamber components linked in serial fashion as to provide a series of linked chamber components comprising first and last serially-linked chamber components;
a plurality of access ports formed along said body, each access port configured to provide access one of said piston chambers formed in said chamber components, respectively;
an inlet comprising a first flow passage to said first serially-linked piston chamber, and a flow passage from said last serially-linked piston chamber;
each said piston chamber formed to receive a modular conditioning component via said respective access port, each said modular conditioning component selected from a collection of modular conditioning components having various fluid regulating characteristics, each said modular conditioning component formed for insertion into and containment via said respective piston chamber and associated access port;
whereby, upon inserting said modular components into said piston chambers and sealing said piston chambers via said respective access port, providing piston chambers having said selected modular conditioning components sealed therein respectively in a desired order, providing a series of linked conditioning components, and facilitating serial flow of fluid therethrough, there is provided a staged conditioning of fluid flowing therethrough via said modular conditioning components.

23. The apparatus of claim 22, wherein said each chamber component is fixed to said adjacent chamber component forming said stack so as to facilitate proper orientation of said piston chamber formed therein, respectively with said respective access port formed in said body, upon slidingly positioning said chamber components in place within said longitudinal receiver.

24. The Apparatus of claim 23, wherein there are further provided alignment an alignment pin situated between said adjacent chamber components forming said stack to fixedly position said chamber components in said proper orientation.

25. The apparatus of claim 24, wherein said modular conditioning components comprise single stage regulators configured for staged pressure reduction of fluid flowing therethrough.
